SEQUENCE LISTING (39 sequences)

SEQ ID NO: 1 - protein, 1368 aa, artificial sequence [sequence omitted]

SEQ ID NO: 2 - DNA, 4104 bp, artificial sequence
  sequence encoding Cas9
atggataaaaaatatagcattggtctggatattggtaccaatagcgttggttgggcagttattaccgatgaatataaagttccgagcaaaaaatttaaagttctgggtaataccgatcgtcatagcattaaaaaaaatctgattggtgcactgctgtttgatagcggtgaaaccgcagaagcaacccgtctgaaacgtaccgcacgtcgtcgttatacccgtcgtaaaaatcgtatttgttatctgcaggaaatttttagcaatgaaatggcaaaagttgatgatagcttttttcatcgtctggaagaaagctttctggttgaagaagataaaaaacatgaacgtcatccgatttttggtaatattgttgatgaagttgcatatcatgaaaaatatccgaccatttatcatctgcgtaaaaaactggttgatagcaccgataaagcagatctgcgtctgatttatctggcactggcacatatgattaaatttcgtggtcattttctgattgaaggtgatctgaatccggataatagcgatgttgataaactgtttattcagctggttcagacctataatcagctgtttgaagaaaatccgattaatgcaagcggtgttgatgcaaaagcaattctgagcgcacgtctgagcaaaagccgtcgtctggaaaatctgattgcacagctgccgggtgaaaaaaaaaatggtctgtttggtaatctgattgcactgagcctgggtctgaccccgaattttaaaagcaattttgatctggcagaagatgcaaaactgcagctgagcaaagatacctatgatgatgatctggataatctgctggcacagattggtgatcagtatgcagatctgtttctggcagcaaaaaatctgagcgatgcaattctgctgagcgatattctgcgtgttaataccgaaattaccaaagcaccgctgagcgcaagcatgattaaacgttatgatgaacatcatcaggatctgaccctgctgaaagcactggttcgtcagcagctgccggaaaaatataaagaaattttttttgatcagagcaaaaatggttatgcaggttatattgatggtggtgcaagccaggaagaattttataaatttattaaaccgattctggaaaaaatggatggtaccgaagaactgctggttaaactgaatcgtgaagatctgctgcgtaaacagcgtacctttgataatggtagcattccgcatcagattcatctgggtgaactgcatgcaattctgcgtcgtcaggaagatttttatccgtttctgaaagataatcgtgaaaaaattgaaaaaattctgacctttcgtattccgtattatgttggtccgctggcacgtggtaatagccgttttgcatggatgacccgtaaaagcgaagaaaccattaccccgtggaattttgaagaagttgttgataaaggtgcaagcgcacagagctttattgaacgtatgaccaattttgataaaaatctgccgaatgaaaaagttctgccgaaacatagcctgctgtatgaatattttaccgtttataatgaactgaccaaagttaaatatgttaccgaaggtatgcgtaaaccggcatttctgagcggtgaacagaaaaaagcaattgttgatctgctgtttaaaaccaatcgtaaagttaccgttaaacagctgaaagaagattattttaaaaaaattgaatgttttgatagcgttgaaattagcggtgttgaagatcgttttaatgcaagcctgggtacctatcatgatctgctgaaaattattaaagataaagattttctggataatgaagaaaatgaagatattctggaagatattgttctgaccctgaccctgtttgaagatcgtgaaatgattgaagaacgtctgaaaacctatgcacatctgtttgatgataaagttatgaaacagctgaaacgtcgtcgttataccggttggggtcgtctgagccgtaaactgattaatggtattcgtgataaacagagcggtaaaaccattctggattttctgaaaagcgatggttttgcaaatcgtaattttatgcagctgattcatgatgatagcctgacctttaaagaagatattcagaaagcacaggttagcggtcagggtgatagcctgcatgaacatattgcaaatctggcaggtagcccggcaattaaaaaaggtattctgcagaccgttaaagttgttgatgaactggttaaagttatgggtcgtcataaaccggaaaatattgttattgaaatggcacgtgaaaatcagaccacccagaaaggtcagaaaaatagccgtgaacgtatgaaacgtattgaagaaggtattaaagaactgggtagccagattctgaaagaacatccggttgaaaatacccagctgcagaatgaaaaactgtatctgtattatctgcagaatggtcgtgatatgtatgttgatcaggaactggatattaatcgtctgagcgattatgatgttgatcatattgttccgcagagctttctgaaagatgatagcattgataataaagttctgacccgtagcgataaaaatcgtggtaaaagcgataatgttccgagcgaagaagttgttaaaaaaatgaaaaattattggcgtcagctgctgaatgcaaaactgattacccagcgtaaatttgataatctgaccaaagcagaacgtggtggtctgagcgaactggataaagcaggttttattaaacgtcagctggttgaaacccgtcagattaccaaacatgttgcacagattctggatagccgtatgaataccaaatatgatgaaaatgataaactgattcgtgaagttaaagttattaccctgaaaagcaaactggttagcgattttcgtaaagattttcagttttataaagttcgtgaaattaataattatcatcatgcacatgatgcatatctgaatgcagttgttggtaccgcactgattaaaaaatatccgaaactggaaagcgaatttgtttatggtgattataaagtttatgatgttcgtaaaatgattgcaaaaagcgaacaggaaattggtaaagcaaccgcaaaatattttttttatagcaatattatgaatttttttaaaaccgaaattaccctggcaaatggtgaaattcgtaaacgtccgctgattgaaaccaatggtgaaaccggtgaaattgtttgggataaaggtcgtgattttgcaaccgttcgtaaagttctgagcatgccgcaggttaatattgttaaaaaaaccgaagttcagaccggtggttttagcaaagaaagcattctgccgaaacgtaatagcgataaactgattgcacgtaaaaaagattgggatccgaaaaaatatggtggttttgatagcccgaccgttgcatatagcgttctggttgttgcaaaagttgaaaaaggtaaaagcaaaaaactgaaaagcgttaaagaactgctgggtattaccattatggaacgtagcagctttgaaaaaaatccgattgattttctggaagcaaaaggttataaagaagttaaaaaagatctgattattaaactgccgaaatatagcctgtttgaactggaaaatggtcgtaaacgtatgctggcaagcgcaggtgaactgcagaaaggtaatgaactggcactgccgagcaaatatgttaattttctgtatctggcaagccattatgaaaaactgaaaggtagcccggaagataatgaacagaaacagctgtttgttgaacagcataaacattatctggatgaaattattgaacagattagcgaatttagcaaacgtgttattctggcagatgcaaatctggataaagttctgagcgcatataataaacatcgtgataaaccgattcgtgaacaggcagaaaatattattcatctgtttaccctgaccaatctgggtgcaccggcagcatttaaatattttgataccaccattgatcgtaaacgttataccagcaccaaagaagttctggatgcaaccctgattcatcagagcattaccggtctgtatgaaacccgtattgatctgagccagctgggtggtgat

SEQ ID NO: 3 - protein, 1082 aa, Geobacillus thermodenitrificans T12
MKYKIGLDIGITSIGWAVINLDIPRIEDLGVRIFDRAENPKTGESLALPRRLARSARRRLRRRKHRLERIRRLFVREGILTKEELNKLFEKKHEIDVWQLRVEALDRKLNNDELARILLHLAKRRGFRSNRKSERTNKENSTMLKHIEENQSILSSYRTVAEMVVKDPKFSLHKRNKEDNYTNTVARDDLEREIKLIFAKQREYGNIVCTEAFEHEYISIWASQRPFASKDDIEKKVGFCTFEPKEKRAPKATYTFQSFTVWEHINKLRLVSPGGIRALTDDERRLIYKQAFHKNKITFHDVRTLLNLPDDTRFKGLLYDRNTTLKENEKVRFLELGAYHKIRKAIDSVYGKGAAKSFRPIDFDTFGYALTMFKDDTDIRSYLRNEYEQNGKRMENLADKVYDEELIEELLNLSFSKFGHLSLKALRNILPYMEQGEVYSTACERAGYTFTGPKKKQKTVLLPNIPPIANPVVMRALTQARKVVNAIIKKYGSPVSIHIELARELSQSFDERRKMQKEQEGNRKKNETAIRQLVEYGLTLNPTGLDIVKFKLWSEQNGKCAYSLQPIEIERLLEPGYTEVDHVIPYSRSLDDSYTNKVLVLTKENREKGNRTPAEYLGLGSERWQQFETFVLTNKQFSKKKRDRLLRLHYDENEENEFKNRNLNDTRYISRFLANFIREHLKFADSDDKQKVYTVNGRITAHLRSRWNFNKNREESNLHHAVDAAIVACTTPSDIARVTAFYQRREQNKELSKKTDPQFPQPWPHFADELQARLSKNPKESIKALNLGNYDNEKLESLQPVFVSRMPKRSITGAAHQETLRRYIGIDERSGKIQTVVKKKLSEIQLDKTGHFPMYGKESDPRTYEAIRQRLLEHNNDPKKAFQEPLYKPKKNGELGPIIRTIKIIDTTNQVIPLNDGKTVAYNSNIVRVDVFEKDGKYYCVPIYTIDMMKGILPNKAIEPNKPYSEWKEMTEDYTFRFSLYPNDLIRIEFPREKTIKTAVGEEIKIKDLFAYYQTIDSSNGGLSLVSHDNNFSLRSIGSRTLKRFEKYQVDVLGNIYKVRGEKRVGVASSSHSKAGETIRPL

SEQ ID NO: 4 - protein, 1300 aa, artificial sequence
  FnCpfI
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 5 - DNA, 3900 bp, artificial sequence
  sequence encoding FnCpfI
atgagcatttatcaggaatttgttaataaatatagcctgagcaaaaccctgcgttttgaactgattccgcagggtaaaaccctggaaaatattaaagcacgtggtctgattctggatgatgaaaaacgtgcaaaagattataaaaaagcaaaacagattattgataaatatcatcagttttttattgaagaaattctgagcagcgtttgtattagcgaagatctgctgcagaattatagcgatgtttattttaaactgaaaaaaagcgatgatgataatctgcagaaagattttaaaagcgcaaaagataccattaaaaaacagattagcgaatatattaaagatagcgaaaaatttaaaaatctgtttaatcagaatctgattgatgcaaaaaaaggtcaggaaagcgatctgattctgtggctgaaacagagcaaagataatggtattgaactgtttaaagcaaatagcgatattaccgatattgatgaagcactggaaattattaaaagctttaaaggttggaccacctattttaaaggttttcatgaaaatcgtaaaaatgtttatagcagcaatgatattccgaccagcattatttatcgtattgttgatgataatctgccgaaatttctggaaaataaagcaaaatatgaaagcctgaaagataaagcaccggaagcaattaattatgaacagattaaaaaagatctggcagaagaactgacctttgatattgattataaaaccagcgaagttaatcagcgtgtttttagcctggatgaagtttttgaaattgcaaattttaataattatctgaatcagagcggtattaccaaatttaataccattattggtggtaaatttgttaatggtgaaaataccaaacgtaaaggtattaatgaatatattaatctgtatagccagcagattaatgataaaaccctgaaaaaatataaaatgagcgttctgtttaaacagattctgagcgataccgaaagcaaaagctttgttattgataaactggaagatgatagcgatgttgttaccaccatgcagagcttttatgaacagattgcagcatttaaaaccgttgaagaaaaaagcattaaagaaaccctgagcctgctgtttgatgatctgaaagcacagaaactggatctgagcaaaatttattttaaaaatgataaaagcctgaccgatctgagccagcaggtttttgatgattatagcgttattggtaccgcagttctggaatatattacccagcagattgcaccgaaaaatctggataatccgagcaaaaaagaacaggaactgattgcaaaaaaaaccgaaaaagcaaaatatctgagcctggaaaccattaaactggcactggaagaatttaataaacatcgtgatattgataaacagtgtcgttttgaagaaattctggcaaattttgcagcaattccgatgatttttgatgaaattgcacagaataaagataatctggcacagattagcattaaatatcagaatcagggtaaaaaagatctgctgcaggcaagcgcagaagatgatgttaaagcaattaaagatctgctggatcagaccaataatctgctgcataaactgaaaatttttcatattagccagagcgaagataaagcaaatattctggataaagatgaacatttttatctggtttttgaagaatgttattttgaactggcaaatattgttccgctgtataataaaattcgtaattatattacccagaaaccgtatagcgatgaaaaatttaaactgaattttgaaaatagcaccctggcaaatggttgggataaaaataaagaaccggataataccgcaattctgtttattaaagatgataaatattatctgggtgttatgaataaaaaaaataataaaatttttgatgataaagcaattaaagaaaataaaggtgaaggttataaaaaaattgtttataaactgctgccgggtgcaaataaaatgctgccgaaagttttttttagcgcaaaaagcattaaattttataatccgagcgaagatattctgcgtattcgtaatcatagcacccataccaaaaatggtagcccgcagaaaggttatgaaaaatttgaatttaatattgaagattgtcgtaaatttattgatttttataaacagagcattagcaaacatccggaatggaaagattttggttttcgttttagcgatacccagcgttataatagcattgatgaattttatcgtgaagttgaaaatcagggttataaactgacctttgaaaatattagcgaaagctatattgatagcgttgttaatcagggtaaactgtatctgtttcagatttataataaagattttagcgcatatagcaaaggtcgtccgaatctgcataccctgtattggaaagcactgtttgatgaacgtaatctgcaggatgttgtttataaactgaatggtgaagcagaactgttttatcgtaaacagagcattccgaaaaaaattacccatccggcaaaagaagcaattgcaaataaaaataaagataatccgaaaaaagaaagcgtttttgaatatgatctgattaaagataaacgttttaccgaagataaatttttttttcattgtccgattaccattaattttaaaagcagcggtgcaaataaatttaatgatgaaattaatctgctgctgaaagaaaaagcaaatgatgttcatattctgagcattgatcgtggtgaacgtcatctggcatattataccctggttgatggtaaaggtaatattattaaacaggatacctttaatattattggtaatgatcgtatgaaaaccaattatcatgataaactggcagcaattgaaaaagatcgtgatagcgcacgtaaagattggaaaaaaattaataatattaaagaaatgaaagaaggttatctgagccaggttgttcatgaaattgcaaaactggttattgaatataatgcaattgttgtttttgaagatctgaattttggttttaaacgtggtcgttttaaagttgaaaaacaggtttatcagaaactggaaaaaatgctgattgaaaaactgaattatctggtttttaaagataatgaatttgataaaaccggtggtgttctgcgtgcatatcagctgaccgcaccgtttgaaacctttaaaaaaatgggtaaacagaccggtattatttattatgttccggcaggttttaccagcaaaatttgtccggttaccggttttgttaatcagctgtatccgaaatatgaaagcgttagcaaaagccaggaattttttagcaaatttgataaaatttgttataatctggataaaggttattttgaatttagctttgattataaaaattttggtgataaagcagcaaaaggtaaatggaccattgcaagctttggtagccgtctgattaattttcgtaatagcgataaaaatcataattgggatacccgtgaagtttatccgaccaaagaactggaaaaactgctgaaagattatagcattgaatatggtcatggtgaatgtattaaagcagcaatttgtggtgaaagcgataaaaaattttttgcaaaactgaccagcgttctgaataccattctgcagatgcgtaatagcaaaaccggtaccgaactggattatctgattagcccggttgcagatgttaatggtaatttttttgatagccgtcaggcaccgaaaaatatgccgcaggatgcagatgcaaatggtgcatatcatattggtctgaaaggtctgatgctgctgggtcgtattaaaaataatcaggaaggtaaaaaactgaatctggttattaaaaatgaagaatattttgaatttgttcagaatcgtaataat

SEQ ID NO: 6 - protein, 1263 aa, Eubacterium rectale
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 7 - protein, 1274 aa, artificial sequence
  MAD7-NLS
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYLSGGSPKKKRKV

SEQ ID NO: 8 - DNA, 1050 bp, artificial sequence
  Lambda genome
aatcacgctgatttacagcggcagccataaggtggatggcaacccctacagccatcttccggatgacgtccgggagacactgcagtcccggatggacgcaacccgccagatgtttgcgcagaaggtgtcggcatataccggcctgtccgtgcaggttgtgctggataccgaggctgcagtgtacagcggtcaggaggccattgatgccggactggctgatgaacttgttaacagcaccgatgcgatcaccgtcatgcgtgatgcactggatgcacgtaaatcccgtctctcaggagggcgaatgaccaaagagactcaatcaacaactgtttcagccactgcttcgcaggctgacgttactgacgtggtgccagcgacggagggcgagaacgccagcgcggcgcagccggacgtgaacgcgcagatcaccgcagcggttgcggcagaaaacagccgcattatggggatcctcaactgtgaggaggctcacggacgcgaagaacaggcacgcgtgctggcagaaacccccggtatgaccgtgaaaacggcccgccgcattctggccgcagcaccacagagtgcacaggcgcgcagtgacactgcgctggatcgtctgatgcagggggcaccggcaccgctggctgcaggtaacccggcatctgatgccgttaacgatttgctgaacacaccagtgtaagggatgtttatgacgagcaaagaaacctttacccattaccagccgcagggcaacagtgacccggctcataccgcaaccgcgcccggcggattgagtgcgaaagcgcctgcaatgaccccgctgatgctggacacctccagccgtaagctggttgcgtgggatggcaccaccgacggtgctgccgttggcattcttgcggttgctgctgaccagaccagcaccacgctgacgttctacaagtccggcacgttccgttatgaggatgtgctctggccggaggctgccagcgacgagacgaaaaaacggaccgcgtttgccggaacggcaatcagcatcgtttaactttacccttc

SEQ ID NO: 9 - DNA, 55 bp, artificial sequence
  Fig. 2. ONT barcode primer B2
aaggttacagacgactacaaacggaatcgaacagcacctgacgatgagtcctgag

SEQ ID NO: 10 - RNA, 34 nt, artificial sequence
  Fig. 2. Adapter RevsgRNA
gacgaugaguccugaguccggaugacguccggga

SEQ ID NO: 11 - DNA, 17 bp, artificial sequence
  Fig. 2. RevsgRNA
ctgcaggccctctgtga

SEQ ID NO: 12 - DNA, 17 bp, artificial sequence
  Fig. 2. sgRNA 3
gctcataccgcaaccgc

SEQ ID NO: 13 - RNA, 35 nt, artificial sequence
  Fig. 2. Adapter sgRNA 3
uggcguuggcgcgggccgccaugcgucagaugcuc

SEQ ID NO: 14 - DNA, 56 bp, artificial sequence
  Fig. 2. ONT Barcode primer B1
ccatgcgtcagatgctctccacgacattctttcaacagccacagaaacacttggaa

SEQ ID NO: 15 - DNA, 16 bp, artificial sequence
  Fig. 2. Primer
gacgatgagtcctgag

SEQ ID NO: 16 - DNA, 18 bp, artificial sequence
  Fig. 2. Primer
gtcgctggcaccacgtca

SEQ ID NO: 17 - DNA, 19 bp, artificial sequence
  Fig. 2. Primer
cactgcgctggatcgtctg

SEQ ID NO: 18 - DNA, 17 bp, artificial sequence
  Fig. 2. Primer
ctcgtagactgcgtacc

SEQ ID NO: 19 - DNA, 21 bp, artificial sequence
  Fig. 2. Primer
acgactacaaacggaatcgaa

SEQ ID NO: 20 - DNA, 18 bp, artificial sequence
  Fig. 2. Primer
gtcgctggcaccacgtca

SEQ ID NO: 21 - DNA, 19 bp, artificial sequence
  Fig. 2. Primer
cactgcgctggatcgtctg

SEQ ID NO: 22 - DNA, 22 bp, artificial sequence
  Fig. 2. Primer
cacaaagacaccgacaactttc

SEQ ID NO: 23 - DNA, 21 bp, artificial sequence
  Fig. 2. Primer
acgactacaaacggaatcgaa

SEQ ID NO: 24 - DNA, 22 bp, artificial sequence
  Fig. 2. Primer
cacaaagacaccgacaactttc

SEQ ID NO: 25 - DNA, 21 bp, artificial sequence
  primer
tcacgctgatttacagcggca

SEQ ID NO: 26 - DNA, 20 bp, artificial sequence
  primer
ggcttgccgttagtcgtagc

SEQ ID NO: 27 - RNA, 103 nt, artificial sequence
  RevsgRNA
agugucucccggacgucaucguuuuagagcuagaaauagcaaguuaaaauaaggcuaguccguuaucaacuugaaaaaguggcaccgagucggugcuuuuuuu

SEQ ID NO: 28 - RNA, 103 nt, artificial sequence
  sgRNA3
gcucauaccgcaaccgcgccguuuuagagcuagaaauagcaaguuaaaauaaggcuaguccguuaucaacuugaaaaaguggcaccgagucggugcuuuuuuu

SEQ ID NO: 29 - RNA, 34 nt, artificial sequence; identical to SEQ ID NO: 10
  RevsgRNA-RNA-Ad

SEQ ID NO: 30 - RNA, 35 nt, artificial sequence
  sgRNA3-RNA-Ad
cucguagacugcguaccgccgggcgcgguugcggu

SEQ ID NO: 31 - DNA, 55 bp, artificial sequence; identical to SEQ ID NO: 9
  RevsgRNA-BC2

SEQ ID NO: 32 - DNA, 56 bp, artificial sequence
  sgRNA3-BC1
aaggttcacaaagacaccgacaactttcttacagcacctctcgtagactgcgtacc

SEQ ID NO: 33 - DNA, 16 bp, artificial sequence
  primer
gacgatgagtcctgag

SEQ ID NO: 34 - DNA, 17 bp, artificial sequence
  primer
ctcgtagactgcgtacc

SEQ ID NO: 35 - DNA, 21 bp, artificial sequence
  primer
acgactacaaacggaatcgaa

SEQ ID NO: 36 - DNA, 22 bp, artificial sequence
  primer
cacaaagacaccgacaactttc

SEQ ID NO: 37 - DNA, 22 bp, artificial sequence
  protelomerase recognition sequence
  misc_feature: n is a, c, g or t
  misc_feature (1)..(1): n is a, c, g, or t
  misc_feature (5)..(6): n is a, c, g or t
  misc_feature (9)..(10): n is a, c, g or t
  misc_feature (13)..(14): n is a, c, g or t
  misc_feature (17)..(18): n is a, c, g or t
  misc_feature (22)..(22): n is a, c, g or t
ncatnntanncgnntannatgn

SEQ ID NO: 38 - DNA, 56 bp, artificial sequence
  protelomerase recognition sequence
tatcagcacacaattgcccattatacgcgcgtataatggactattgtgtgctgata

SEQ ID NO: 39 - protein, 631 aa, artificial sequence
  protelomerase
MSKVKIGELINTLVNEVEAIDASDRPQGDKTKRIKAAAARYKNALFNDKRKFRGKGLQKRITANTFNAYMSRARKRFDDKLHHSFDKNINKLSEKYPLYSEELSSWLSMPTANIRQHMSSLQSKLKEIMPLAEELSNVRIGSKGSDAKIARLIKKYPDWSFALSDLNSDDWKERRDYLYKLFQQGSALLEELHQLKVNHEVLYHLQLSPAERTSIQQRWADVLREKKRNVVVIDYPTYMQSIYDILNNPATLFSLNTRSGMAPLAFALAAVSGRRMIEIMFQGEFAVSGKYTVNFSGQAKKRSEDKSVTRTIYTLCEAKLFVELLTELRSCSAASDFDEVVKGYGKDDTRSENGRINAILAKAFNPWVKSFFGDDRRVYKDSRAIYARIAYEMFFRVDPRWKNVDEDVFFMEILGHDDENTQLHYKQFKLANFSRTWRPEVGDENTRLVALQKLDDEMPGFARGDAGVRLHETVKQLVEQDPSAKITNSTLRAFKFSPTMISRYLEFAADALGQFVGENGQWQLKIETPAIVLPDEESVETIDEPDDESQDDELDEDEIELDEGGGDEPTEEEGPEEHQPTALKPVFKPAKNNGDGTYKIEFEYDGKHYAWSGPADSPMAAMRSAWETYYS

(19) United States
(12) Patent Application Publication
(10) Pub. No.: US 2023/0407366 A1
(43) Pub. Date: Dec. 21, 2023

(54) TARGETED SEQUENCE ADDITION

(71) Applicant: KEYGENE N.V., AE Wageningen (NL)

(72) Inventors: René Cornelis Josephus HOGERS, AE Wageningen (NL); Stefan John WHITE, AE Wageningen (NL); Theodorus Frank Maria ROELOFS, AE Wageningen (NL)

(73) Assignee: KEYGENE N.V., AE Wageningen (NL)

(21) Appl. No.: 18/029,493

(22) PCT Filed: Oct. 6, 2021
(86) PCT No.: PCT/EP2021/077567

§ 371 Date: Mar. 30, 2023

Related U.S. Application Data
(60) Provisional application No. 63/118,781, filed on Nov. 27, 2020.

(30) Foreign Application Priority Data
Oct. 6, 2020 (EP) 20200254.9

(51) Int. Cl. C12Q 1/6806; C12Q 1/48; C12N 9/22; C12N 15/10

(52) U.S. Cl. CPC C12Q 1/6806; C12Q 1/485; C12N 2310/20; C12N 15/1093; C12N 9/22

(57) ABSTRACT

The invention pertains to a method for labelling a target nucleic acid fragment using a combination of a site-specific nuclease and a reverse transcriptase. The labelling results in the addition of a specific nucleotide sequence to at least one free 3′-end of the target nucleic acid fragment. The invention further relates to a method for determining the sequence of the target nucleic acid fragment as well as construct and kit for use in the method of the invention.

### FIELD OF THE INVENTION

The present invention is in the field of genetic research, more particular in the field of targeted nucleic acid isolation, e.g. for library preparation for further analysis or processing in genetic research. Disclosed are new methods and compositions for complexity reduction of nucleic acid samples or enrichment of target nucleic acids within nucleic acid samples.

### BACKGROUND

A significant component of genetic research is sequence analysis of defined DNA loci. This can be to genotype known variants, or identify sequence changes or variants. Such analysis often needs to be done in a multiplex context, e.g., a specific set of loci needs to be analyzed in a large number of samples. The ideal assay to do this is flexible with regard to the number of samples and loci that need to be screened, is highly accurate, and is amenable to different sequencing platforms. Attempts have been made to provide for assays that comprise an enrichment step but are ideally amplification free. For instance, US2014/0134610 describes a complexity reduction method using type II restriction enzymes to fragment nucleic acids in a sample, followed by ligation of protective adapters and subsequently degrading all non-captured nucleic acid using exonucleases. In WO2016/028887, this method is amended by using a programmable endonuclease, i.e. a CRISPR-endonuclease for fragmenting the nucleic acid in the sample.

CRISPRs (Clustered Regularly Interspaced Short Palindromic Repeats) are loci containing multiple short direct repeats and are found in 40% of the sequenced bacteria and 90% of sequenced archaea. The CRISPR repeats form a system of acquired bacterial immunity against genetic pathogens such as bacteriophages and plasmids. When a bacterium is challenged with a pathogen, a small piece of the pathogen's genome is processed by CRISPR associated proteins (CAS) and incorporated into the bacterial genome between CRISPR repeats. The CRISPR loci are then transcribed and processed to form so called crRNAs which include approximately 30 bps of sequence identical to the pathogen's genome. These RNA molecules form the basis for the recognition of the pathogen upon a subsequent infection and lead to silencing of the pathogen's genetic elements through direct digestion of the pathogen's genome. The CAS protein Cas9 is an essential component of the type-II CRISPR-CAS system from S. pyogenes and forms an endonuclease, when combined with the crRNA together with a second RNA termed the trans-activating crRNA (tracrRNA). This complex targets the invading pathogenic DNA for degradation by the introduction of DNA double strand breaks (DSBs) at the position in the genome defined by the crRNA. This type-II CRISPR-Cas9 system has been proven to be a convenient and effective tool in biochemistry that, via the targeted introduction of double-strand breaks and the subsequent activation of endogenous repair mechanisms, is capable of introducing modification in eukaryotic genomes at sites of interest. Jinek et al. (2012, Science 337: 816-820) demonstrated that a single chain chimeric RNA (single guide RNA, sRNA, sgRNA), produced by combining the essential sequences of the crRNA and tracrRNA into a single RNA molecule, was able to form a functional endonuclease in combination with Cas9. Since then, many different CRISPR-CAS systems have been identified from different bacterial species (Zetsche et al. 2015 Cell 163, 759-771; Kim et al. 2017, Nat. Commun. 8, 1-7; Ran et al. 2015. Nature 520, 186-191).

Besides CRISPR-CAS systems, in which RNA guides are used to direct an endonuclease to a specific position in a nucleic acid molecule, other endonucleases are known in the art which use DNA or RNA guides (Doxzen et al. 2017, PLOS ONE 12(5): e0177097; Kaya et al. 2016, PNAS vol. 113 no. 15, 4057-4062).

Recently, the CRISPR system has been used to specifically edit DNA in a process called “prime editing” (Anzalone AV et al, Nature. 2019; 576(7785): 149-157). Using a catalytically impaired Cas9 endonuclease fused to an engineered reverse transcriptase, specific edits could be made at pre-determined genomic locations.

There is still a strong need in the art for a versatile and accurate method for nucleic acid complexity reduction. In particular, there is a need in the art for a method that allows for flexible and effective labelling of nucleic acid molecules, e.g. for subsequent analysis or processing in genetic research.

The present invention, described in detail below, allows for a versatile method of library preparation for downstream processing and/or analysis.

### SUMMARY

The invention may be summarized in the following embodiments:

Embodiment 1. A method for labelling a target nucleic acid fragment, wherein the target nucleic acid fragment comprises a first strand and a complementary second strand and wherein the target nucleic acid fragment comprises a sequence of interest, wherein the method comprises the steps of:
a) providing a sample comprising a double-stranded nucleic acid molecule, wherein the double-stranded nucleic acid molecule comprises the sequence of interest;
b) contacting the double-stranded nucleic acid molecule with a site-specific nuclease to generate a double-stranded break, wherein the double-stranded break results in a free 3′-end of the first strand of the target nucleic acid fragment; and
c) contacting the cleaved nucleic acid molecule with a reverse transcriptase and a template

RNA molecule, thereby labelling the free 3′-end of the first strand of the target nucleic acid fragment with one or more nucleotides,
wherein optionally the site-specific nuclease in step b) and the reverse transcriptase in step c) are separate entities.

Embodiment 2. The method according to embodiment 1, wherein the method further comprises a step of:
d) contacting the double-stranded nucleic acid molecule with a second site-specific nuclease to generate a second double-stranded break, wherein the second double-stranded break results in a free 3′-end of the second strand of the target nucleic acid fragment, wherein preferably step d) is performed simultaneously with step b).

Embodiment 3. The method according to embodiment 2, wherein the method further comprises a step of:
e) contacting the target nucleic acid fragment with a reverse transcriptase and a second template RNA molecule, thereby labelling the second strand of the target nucleic acid fragment at the free 3′-end with one or more nucleotides, wherein preferably step e) is performed simultaneously with step c).

Embodiment 4. The method according to any one of the preceding embodiments, wherein the site-specific nuclease in step b) and/or step d) is a CRISPR-nuclease complex, preferably comprising at least one of a Cas9 or Cpf1 nuclease.

Embodiment 5. The method according to embodiment 4, wherein the CRISPR-nuclease complex comprises a crRNA and optionally a tracrRNA.

Embodiment 6. The method according to embodiment 4 or 5, wherein the template RNA molecule of step c) comprises a sequence at its 3′ end that can anneal to a sequence at the 3′ end of the first strand of the target nucleic acid fragment, and wherein optionally the sequence at the 3′ end of the template RNA molecule is partly or fully complementary to the sequence of the crRNA of the cite-specific nuclease in step b).

Embodiment 7. The method according to any one of embodiments 4-6, wherein the template RNA molecule of step e) comprises a sequence at its 3′ end that can anneal to a sequence at the 3′ end of the second strand of the target nucleic acid fragment, and wherein optionally the sequence at the 3′ end of the template RNA molecule is partly or fully complementary to the sequence of the crRNA of the site-specific nuclease in step d).

Embodiment 8. The method according to any one of embodiments 4-7, wherein the template RNA and the crRNA, and optionally the tracrRNA, are separate RNA molecules.

Embodiment 9. The method according to any one of the preceding embodiments, wherein the sequence of the nucleotides extending the first strand differs from the sequence of the nucleotides extending the second strand of the target nucleic acid fragment, wherein preferably the one or more nucleotides extending the first and second strand have less than 90%, 80%, 60% or less than 40% nucleotide sequence identity.

Embodiment 10. The method according to any one of the preceding embodiments, wherein the method further comprises a step of:
f) annealing a first oligonucleotide to the labelled 3′-end of the first strand of the target nucleic acid fragment, wherein optionally the template RNA and crRNA are degraded prior to annealing the first oligonucleotide.

Embodiment 11. The method according to embodiment 10, wherein the oligonucleotide annealing to the labelled 3′-end of the first strand is not capable of annealing to the, optionally labelled, 3′-end of the second strand under normal hybridizing conditions.

Embodiment 12. The method according to any one of embodiment 10 or 11, wherein step f) further comprises annealing a second oligonucleotide to the labelled 3′-end of the second strand, wherein preferably the oligonucleotide annealing to the labelled 3′-end of the second strand is not capable of annealing to the, optionally labelled, 3′-end of the first strand under normal hybridizing conditions.

Embodiment 13. The method according to any one of embodiments 10-12, wherein the method further comprises a step of:
g) ligating and/or filling in the annealed oligonucleotide(s).

Embodiment 14. The method according to any one of embodiments 10-13, wherein at least one of the first and second oligonucleotide comprises at least one of an UMI, a barcode and a primer binding site.

Embodiment 15. A method for sequencing, preferably deep-sequencing, one or more target nucleic acid fragments, comprising the steps of:
(i) obtaining one or more labelled target nucleic acid fragments as defined in any one of embodiments 1-14;
(ii) optionally amplifying, preferably selectively amplifying, the one or more labelled target nucleic acid fragments; and
(iii) determining at least part of the sequence of the, optionally amplified, one or more target nucleic acid fragments.

Embodiment 16. The method according to embodiment 15, wherein the one or more target nucleic acid fragments are obtained from one or more nucleic acid samples, and wherein optionally the one or more target nucleic acid fragments are pooled after step (i) and/or after step (ii).

Embodiment 17. A labelled target nucleic acid fragment obtainable by the method according to any one of embodiments 1-14 or a deep-sequencing library obtainable by the method according to embodiment 15 or 16.

Embodiment 18. A construct encoding a site-specific nuclease and at least one of a reverse transcriptase and a template RNA molecule for use in a method according to any one of embodiments 1-16.

Embodiment 19. The construct according to embodiment 18, further encoding a crRNA and optionally a tracrRNA.

Embodiment 20. A kit of parts comprising at least a first, second and third component for use in a method according to any one of embodiments 1-16, wherein:
the first component is a site-specific nuclease, or construct encoding the same, and optionally at least one of a crRNA, tracrRNA and a sgRNA, or construct encoding the same;
the second component is a reverse transcriptase, or construct encoding the same; and
the third component is a template RNA molecule, or construct encoding the same.

Embodiment 21. The kit of parts according to embodiment 20, wherein the kit further comprises at least one of a fourth, fifth, sixth and seventh component, wherein
the fourth component is one or more oligonucleotides as defined in any one of embodiments 10-14, wherein the one or more oligonucleotides optionally comprise at least one of a UMI, a barcode and a primer binding site;
the fifth component is one or more primers for amplification of a labelled target nucleic acid fragment as defined in embodiment 15;
the sixth component is one or more primers for non-selective amplification of the labelled target nucleic acid fragment; and
the seventh component is one or more primers for selective amplification of a subset of target nucleic acid fragments.

### Definitions

Various terms relating to the methods, compositions, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein. Although any methods and materials similar or equivalent to those described herein can be used in the practice for testing of the present invention, the preferred materials and methods are described herein.

Methods of carrying out the conventional techniques used in methods of the invention will be evident to the skilled worker. The practice of conventional techniques in molecular biology, biochemistry, computational chemistry, cell culture, recombinant DNA, bioinformatics, genomics, sequencing and related fields are well-known to those of skill in the art and are discussed, for example, in the following literature references: Sambrook et al. Molecular Cloning. A Laboratory Manual, 2nd Edition, Cold Spring Harbor Laboratory Press, Cold Spring Harbor, N. Y., 1989; Ausubel et al. Current Protocols in Molecular Biology, John Wiley & Sons, New York, 1987 and periodic updates; and the series Methods in Enzymology, Academic Press, San Diego.

“A,” “an,” and “the”: these singular form terms include plural referents unless the content clearly dictates otherwise. Thus, for example, reference to “a cell” includes a combination of two or more cells, and the like.

As used herein, the term “about” is used to describe and account for small variations. For example, the term can refer to less than or equal to ±10%, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

“And/or”: the term “and/or” refers to a situation wherein one or more of the stated cases may occur, alone or in combination with at least one of the stated cases, up to with all of the stated cases.

As used herein, the term “adapter” is a single-stranded, double-stranded, partly double-stranded, Y-shaped or hairpin nucleic acid molecule that can be attached, preferably ligated, to the end of other nucleic acids, e.g., to a single strand or both strands of a double-stranded DNA molecule, and preferably has a limited length, e.g., about 10 to about 200, or about 10 to about 100 bases, or about 10 to about 80, or about 10 to about 50, or about 10 to about 30 base pairs in length, and is preferably chemically synthesized. The double-stranded structure of the adapter may be formed by two distinct oligonucleotide molecules that are base paired with one another, or by a hairpin structure of a single oligonucleotide strand. As would be apparent, the attachable end of an adapter may be designed to be compatible with, and optionally able to ligate to, overhangs made by cleavage by a restriction enzyme and/or programmable nuclease, may be designed to be compatible with an overhang created after addition of a non-template elongation reaction (e.g. using the method as defined herein), or may have blunt ends. Optionally, the fully or partially double-stranded adapter comprises an overhang, wherein preferably the overhang is a 3′ overhang. Preferably, there is a phosphorothioate bond before the terminal nucleotide. Optionally, the strand opposite to the strand comprising the overhang, is 5′-phosphorylated.

“Amplification” used in reference to a nucleic acid or nucleic acid reactions, refers to in vitro methods of making copies of a particular nucleic acid, such as a target nucleic acid fragment or the sequence of interest comprised in the target nucleic acid fragment. Numerous methods of amplifying nucleic acids are known in the art, and amplification reactions include polymerase chain reactions, ligase chain reactions, strand displacement amplification reactions, rolling circle amplification reactions, transcription-mediated amplification methods such as NASBA (e.g., U.S. Pat. No. 5,409,818), loop mediated amplification methods (e.g., “LAMP” amplification using loop-forming sequences, e.g., as described in U.S. Pat. No. 6,410,278) and isothermal amplification reactions. The nucleic acid that is amplified can be DNA comprising, consisting of, or derived from, DNA or RNA or a mixture of DNA and RNA, including modified DNA and/or RNA. The products resulting from amplification of a nucleic acid molecule or molecules (i.e., “amplification products”), whether the starting nucleic acid is DNA, RNA or both, can be either DNA or RNA, or a mixture of both DNA and RNA nucleosides or nucleotides, or they can comprise modified DNA or RNA nucleosides or nucleotides.

A “copy” can be, but is not limited to, a sequence having full sequence complementarity or full sequence identity to a particular sequence. Alternatively, a copy does not necessarily have perfect sequence complementarity or identity to this particular sequence, e.g. a certain degree of sequence variation is allowed. For example, copies can include nucleotide analogs such as deoxyinosine or deoxyuridine, intentional sequence alterations (such as sequence alterations introduced through a primer comprising a sequence that can be hybridized, but is not complementary, to a particular sequence), and/or sequence errors that occur during amplification.

The term “complementarity” is herein defined as the sequence identity of a sequence to a fully complementary strand (e.g. the second, or reverse, strand). For example, a sequence that is 100% complementary (or fully complementary) is herein understood as having 100% sequence identity with the complementary strand and e.g. a sequence that is 80% complementary is herein understood as having 80% sequence identity to the (fully) complementary strand.

“Comprising”: this term is construed as being inclusive and open ended, and not exclusive. Specifically, the term and variations thereof mean the specified features, steps or components are included. These terms are not to be interpreted to exclude the presence of other features, steps or components.

“Construct” or “nucleic acid construct” or “vector”: this refers to a man-made nucleic acid molecule resulting from the use of recombinant DNA technology and which can be used to deliver exogenous DNA into a host cell, often with the purpose of expression in the host cell of a DNA region comprised on the construct. The vector backbone of a construct may for example be a plasmid into which a (chimeric) gene is integrated or, if a suitable transcription regulatory sequence is already present (for example a (inducible) promoter), only a desired nucleotide sequence (e.g., a coding sequence) is integrated downstream of the transcription regulatory sequence. Vectors may comprise further genetic elements to facilitate their use in molecular cloning, such as e.g., selectable markers, multiple cloning sites and the like.

The terms “double-stranded” and “duplex” as used herein, describes two complementary polynucleotides that are base-paired, i.e., hybridized together. Complementary nucleotide strands are also known in the art as reverse-complement.

The term “effective amount,” as used herein, refers to an amount of a biologically active agent that is sufficient to elicit a desired biological effect. For example, in some embodiments, an effective amount of a site-specific nuclease may refer to the amount of the nuclease that is sufficient to induce cleavage of a double-stranded nucleic acid molecule. As will be appreciated by the skilled artisan, the effective amount of an agent may vary depending on various factors such as the agent being used, the conditions wherein the agent is used, and the desired biological effect, e.g. degree of cleavage to be detected.

“Exemplary”: this terms means “serving as an example, instance, or illustration,” and should not be construed as excluding other configurations disclosed herein.

“Expression”: this refers to the process wherein a DNA region, which is operably linked to appropriate regulatory regions, particularly a promoter, is transcribed into an RNA, which in turn may be translated into a protein or peptide.

A “guide sequence” is to be understood herein as a sequence that directs an RNA or DNA guided endonuclease to a specific site in an RNA or DNA molecule. In the context of a gRNA-CAS complex, “guide sequence” is further to be understood herein as the section of the sgRNA or crRNA, which is required for targeting a gRNA-CAS complex to a specific site in a duplex DNA.

A “qRNA-CAS complex” is to be understood herein as a CAS protein, also named a CRISPR-endonuclease or CRISPR-nuclease, which is complexed or hybridized to a guide RNA, wherein the guide RNA may be a crRNA and/or a tracrRNA, or a sgRNA.

“Identity” and “similarity” can be readily calculated by known methods. “Sequence identity” and “sequence similarity” can be determined by alignment of two peptide or two nucleotide sequences using global or local alignment algorithms, depending on the length of the two sequences. Sequences of similar lengths are preferably aligned using a global alignment algorithm (e.g. Needleman Wunsch) which aligns the sequences optimally over the entire length, while sequences of substantially different lengths are preferably aligned using a local alignment algorithm (e.g. Smith Waterman). Sequences may then be referred to as “substantially identical” or “essentially similar” when they (when optimally aligned by for example the programs GAP or BESTFIT using default parameters) share at least a certain minimal percentage of sequence identity (as defined below). The percent of sequence identity is preferably determined using the “BESTFIT” or “GAP” program of the Sequence Analysis Software Package™ (Version 10; Genetics Computer Group, Inc., Madison, Wis.). GAP uses the Needleman and Wunsch global alignment algorithm (Needleman and Wunsch, Journal of Molecular Biology 48:443-453, 1970) to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, CA 92121-3752 USA, or using open source software, such as the program “needle” (using the global Needleman Wunsch algorithm) or “water” (using the local Smith Waterman algorithm) in EmbossWIN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blosum62 for proteins and DNAFull for DNA). “BESTFIT” performs an optimal alignment of the best segment of similarity between two sequences and inserts gaps to maximize the number of matches using the local homology algorithm of Smith and Waterman (Smith and Waterman, Advances in Applied Mathematics, 2:482-489, 1981, Smith et al., Nucleic Acids Research 11:2205-2220, 1983). When sequences have a substantially different overall lengths, local alignments, such as those using the Smith Waterman algorithm, are preferred.

As used herein “sequence identity” refers to the extent to which two optimally aligned polynucleotide or peptide sequences are invariant throughout a window of alignment of components, e.g., nucleotides or amino acids. An “identity fraction” for aligned segments of a test sequence and a reference sequence is the number of identical components which are shared by the two aligned sequences divided by the total number of components in reference sequence segment, i.e., the entire reference sequence or a smaller defined part of the reference sequence. “Percent identity” is the identity fraction times 100.

Useful methods for determining sequence identity are also disclosed in Guide to Huge Computers, Martin J. Bishop, ed., Academic Press, San Diego, 1994, and Carillo, H., and Lipton, D., Applied Math (1988) 48:1073. More particularly, preferred computer programs for determining sequence identity include the Basic Local Alignment Search Tool (BLAST) programs which are publicly available from National Center Biotechnology Information (NCBI) at the National Library of Medicine, National Institute of Health, Bethesda, Md. 20894; see BLAST Manual, Altschul et al., NCBI, NLM, NIH; Altschul et al., J. Mol. Biol. 215:403-410 (1990); version 2.0 or higher of BLAST programs allows the introduction of gaps (deletions and insertions) into alignments; for peptide sequence BLASTX can be used to determine sequence identity; and, for polynucleotide sequence BLASTN can be used to determine sequence identity.

Alternatively, percentage similarity or identity may be determined by searching against public databases, using algorithms such as FASTA, BLAST, etc. Thus, the nucleic acid and protein sequences of the present invention can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10. BLAST nucleotide searches can be performed with the BLASTn program, expect threshold=0.05, word size=28 to obtain nucleotide sequences homologous to nucleic acid molecules of the invention. BLAST protein searches can be performed with the BLASTx program, expect threshold=0.05, word size=6 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., BLASTx and BLASTn) can be used. See the homepage of the National Center for Biotechnology Information at http://www.ncbi.nlm.nih.gov/.

“Nanopore selective sequencing” is to be understood herein as selectively sequencing of single molecules in real time using nanopore sequencing technology such as from Oxford Nanopore or Ontera, and mapping streaming nanopore current signals or base calls to a reference sequence in order to reject non-target sequences. In response to the data being generated, the sequencer is steered to either pursue sequencing of a nucleic acid, or decides to quit and remove the nucleic acid from the sequencing pore by reversing the polarity of the voltage across the specific pore for a certain short period of time sufficient to eject the non-target molecule and making the nanopore available for a new sequencing read. Examples of Nanopore selective sequencing methods are described in Payne et al., 2020 (Nanopore adaptive sequencing for mixed samples, whole exome capture and targeted panels, Feb. 3, 2020; DOI: 10.1101/2020.02.03.926956) and Kovaka et al. 2020 (Targeted nanopore sequencing by real-time mapping of raw electrical signal with UNCALLED, Feb. 3, 2020; doi: 10.1101/2020.02.03.931923), which are incorporated herein by reference.

The term “nucleotide” includes, but is not limited to, naturally-occurring nucleotides, including guanine, cytosine, adenine and thymine (G, C, A and T, respectively). The term “nucleotide” is further intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the term “nucleotide” includes those moieties that contain hapten or fluorescent labels and may contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The terms “nucleic acid”, “polynucleotide” and “nucleic acid molecule” are used interchangeably herein to describe a polymer of any length, e.g., greater than about 2 bases, greater than about 10 bases, greater than about 100 bases, greater than about 500 bases, greater than 1000 bases, up to about 10,000 or more bases composed of nucleotides, e.g., deoxyribonucleotides or ribonucleotides, and may be produced enzymatically or synthetically (e.g., PNA as described in U.S. Pat. No. 5,948,902 and the references cited therein). The nucleic acid may hybridize with naturally occurring nucleic acids in a sequence specific manner analogous to that of two naturally occurring nucleic acids, e.g., can participate in Watson-Crick base pairing interactions. In addition, nucleic acids and polynucleotides may be isolated (and optionally subsequently fragmented) from cells, tissues and/or bodily fluids. The nucleic acid can be e.g. genomic DNA (gDNA), mitochondrial, cell free DNA (cfDNA), DNA from a library and/or RNA from a library.

The term “nucleic acid sample” or “sample comprising a double-stranded nucleic acid molecule” as used herein denotes any sample containing a nucleic acid molecule, wherein a sample relates to a material or mixture of materials, typically, although not necessarily, in liquid form, containing one or more nucleotide sequences of interest. The nucleic acid sample used as starting material in the method of the invention can be from any source, e.g., a whole genome, a collection of chromosomes, a single chromosome, one or more regions from one or more chromosomes or transcribed genes, and may be purified directly from the biological source or from a laboratory source, e.g., a nucleic acid library. The nucleic acid samples can be obtained from the same individual, which can be a human or other species (e.g., plant, bacteria, fungi, algae, archaea, etc.), or from different individuals of the same species, or different individuals of different species. For example, the nucleic acid samples may be from a cell, tissue, biopsy, bodily fluid, genome DNA library, cDNA library and/or a RNA library.

The term “ sequence of interest” includes, but is not limited to, any genetic sequence preferably present within a cell, such as, for example a gene, part of a gene, or a non-coding sequence within or adjacent to a gene. The target sequence of interest may be present in a chromosome, an episome, an organellar genome such as mitochondrial or chloroplast genome or genetic material that can exist independently to the main body of genetic material such as an infecting viral genome, plasmids, episomes, transposons for example. A sequence of interest may be within the coding sequence of a gene, within transcribed non-coding sequence such as, for example, leader sequences, trailer sequence or introns. Said sequence of interest may be present in a double or a single strand nucleic acid molecule. The nucleic acid sequence is preferably present in a double-stranded nucleic acid molecule. The sequence of interest can be, but is not limited to, a sequence having or suspected of having, a polymorphism, e.g. a SNP. In some embodiments, the sequence of interest is an allelic variant, or the reverse complement thereof. The sequence of interest may be any sequence within a sample nucleic acid, e.g., a gene, gene complex, locus, pseudogene, regulatory region, highly repetitive region, polymorphic region, or portion thereof. The sequence of interest may also be a region comprising genetic or epigenetic variations indicative for a phenotype or disease. Preferably, the sequence of interest is a small or longer contiguous stretch of nucleotides (i.e. a polynucleotide) of a single-strand DNA strand of duplex DNA, wherein said duplex DNA further comprises a sequence complementary to the target sequence in the complementary strand of said duplex DNA. Duplex DNA consisting of the sequence of interest and its complementary strand is also denominated herein as a target nucleic acid fragment.

“Target nucleic acid fragment” may be a small or longer stretch, or selected portion of a nucleic acid molecule, preferably double-stranded, comprising or consisting of a sequence of interest, that is preferably the object of a further analysis or action, such as, but not limited to copying, amplification, sequencing and/or other procedure for nucleic acid interrogation. Prior to cleavage, the target nucleic acid fragment is preferably comprised within a larger nucleic acid molecule, e.g. within a larger nucleic acid molecule present in a sample to be analyzed. The target nucleic acid fragment preferably comprises a first strand and a complementary second strand. In some aspects, a set of target nucleic acid fragments comprising or consisting of one or more sequences of interest are selected to be enriched. Optionally, such set consists of structurally or functionally related target nucleic acid fragments. A target nucleic acid fragment, or fragments, can comprise both natural and non-natural, artificial, or non-canonical nucleotides including, but not limited to, DNA, RNA, BNA (bridged nucleic acid), LNA (locked nucleic acid), PNA (peptide nucleic acid), morpholino nucleic acid, glycol nucleic acid, threose nucleic acid, epigenetically modified nucleotide such as methylated DNA, and mimetics and combinations thereof. Preferably, the target nucleic acid fragment is genomic DNA (gDNA) and/or cell free DNA (cfDNA).

The term “oliqonucleotide” as used herein denotes a single-stranded multimer of nucleotides, preferably of about 2 to 200 nucleotides, or up to 500 nucleotides in length. Oligonucleotides may be synthetic or may be made enzymatically, and, in some embodiments, are about 10 to 50 nucleotides in length. Oligonucleotides may contain ribonucleotide monomers (i.e., may be oligoribonucleotides) or deoxyribonucleotide monomers. An oligonucleotide may be about 10 to 20, to 30, 30 to 40, 40 to 50, 50 to 60, 60 to 70, 70 to 80, 80 to 100, 100 to 150, 150 to 200, or about 200 to 250 nucleotides in length, for example.

“Plant”: this includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, grains and the like. Non-limiting examples of plants include crop plants and cultivated plants, such as barley, cabbage, canola, cassava, cauliflower, chicory, cotton, cucumber, eggplant, grape, hot pepper, lettuce, maize, melon, oilseed rape, potato, pumpkin, rice, rye, sorghum, squash, sugar cane, sugar beet, sunflower, sweet pepper, tomato, water melon, wheat, and zucchini.

The “protospacer sequence” is the sequence that is recognized or can be hybridized to a guide sequence within a guide RNA, more specifically the crRNA or, in case of a sgRNA, the crRNA part of the guide RNA, and is located in, at or near the target nucleic acid fragment.

An “endonuclease” is an enzyme that hydrolyses at least one strand of a duplex DNA or a strand of an RNA molecule, upon binding to its target or recognition site. An endonuclease is to be understood herein as a site-specific endonuclease and the terms “endonuclease” and “nuclease” are used interchangeable herein. A restriction endonuclease is to be understood herein as an endonuclease that hydrolyses both strands of the duplex at the same time to introduce a double strand break in the DNA. A “nicking” endonuclease is an endonuclease that hydrolyses only one strand of the duplex to produce DNA molecules that are “nicked” rather than cleaved.

An “exonuclease” is defined herein as any enzyme that cleaves one or more nucleotides from the end (exo) of a polynucleotide.

“Reducing complexity” or “complexity reduction” is to be understood herein as the reduction of a complex nucleic acid sample, such as samples derived from genomic DNA, cfDNA derived from liquid biopsies, isolated RNA samples and the like. Reduction of complexity results in the enrichment of one or more specific target sequences and/or target nucleic acid fragments comprised within the complex starting material and/or the generation of a subset of the sample, wherein the subset comprises or consists of one or more specific target sequences or fragments comprised within the complex starting material, while non-target sequences or fragments are reduced in amount by at least 20%, 30%, 40%, 50%, 60%, 70%, 80%, 90%, 91%, 92%, 93%, 94%, 95%, 96%, 97%, 98% or 99% as compared to the amount of non-target sequences or fragments in the starting material, i.e. before complexity reduction. Reduction of complexity is in general performed prior to further analysis or method steps, such as amplification, barcoding, sequencing, determining epigenetic variation etc. Preferably, complexity reduction is reproducible complexity reduction, which means that when the same sample is reduced in complexity using the same method, the same, or at least comparable, subset is obtained, as opposed to random complexity reduction. Examples of complexity reduction methods include for example AFLP® (Keygene N.V., the Netherlands; see e.g., EP 0 534 858), Arbitrarily Primed PCR amplification, capture-probe hybridization, the methods described by Dong (see e.g., WO 03/012118, WO 00/24939) and indexed linking (Unrau P. and Deugau K. V. (1994) Gene 145:163-169), the methods described in WO2006/137733; WO2007/037678; WO2007/073165; WO2007/073171, US 2005/260628, WO 03/010328, US 2004/10153, genome portioning (see e.g. WO 2004/022758), Serial Analysis of Gene Expression (SAGE; see e.g. Velculescu et al., 1995, see above, and Matsumura et al., 1999, The Plant Journal, vol. 20 (6): 719-726) and modifications of SAGE (see e.g. Powell, 1998, Nucleic Acids Research, vol. 26 (14): 3445-3446; and Kenzelmann and Mühlennann, 1999, Nucleic Acids Research, vol. 27 (3): 917-918), MicroSAGE (see e.g. Datson et al., 1999, Nucleic Acids Research, vol. 27 (5): 1300-1307), Massively Parallel Signature Seguencing (MPSS; see e.g. Brenner et al., 2000, Nature Biotechnology, vol. 18:630-634 and Brenner et al., 2000, PNAS, vol. 97 (4):1665-1670), self-subtracted cDNA libraries (Laveder et al., 2002, Nucleic Acids Research, vol. Real-Time Multiplex Ligation-dependent Probe Amplification (RT-MLPA; see e.g. Eldering et al., 2003, vol. 31 (23): el53), High Coverage Expression Profiling (HiCEP; see e.g. Fukumura et al., 2003, Nucleic Acids Research, vol. 31(16):e94), a universal micro-array system as disclosed in Roth et al.(Roth et al., 2004, Nature Biotechnology, vol. 22 (4): 418-426), a transcriptome subtraction method (see e.g. Li et al., Nucleic Acids Research, vol. 33 (16): el36), and fragment display (see e.g. Metsis et al., 2004, Nucleic Acids Research, vol. 32 (16): el27).

“Sequence” or “Nucleotide sequence”: This refers to the order of nucleotides of, or within a nucleic acid. In other words, any order of nucleotides in a nucleic acid may be referred to as a sequence or nucleic acid sequence. For example, the target sequence is an order of nucleotides comprised in a single strand of a DNA duplex.

The term “sequencing,” as used herein, refers to a method by which the identity of at least consecutive nucleotides (e.g., the identity of at least 20, at least 50, at least 100 or at least 200 or more consecutive nucleotides) of a polynucleotide are obtained. The terms “next-generation sequencing”, “deep-sequencing” or “high-throughput sequencing” may be used interchangeably herein and refers to the so-called parallelized sequencing-by-synthesis or sequencing-by-ligation platforms, e.g., such as currently employed by Illumina, Life Technologies, PacBio and Roche etc. Next-generation sequencing methods may also include nanopore sequencing methods, such as those commercialized by Oxford Nanopore Technologies, or electronic-detection based methods such as Ion Torrent technology commercialized by Life Technologies. Preferably, the next-generation sequencing method is a nanopore sequencing method, preferably a nanopore selective sequencing method.

A “unique molecular identifier” or “UMI” is a substantially unique tag (e.g. barcode), preferably fully unique, that is specific for a nucleic acid molecule, e.g. unique for each single polynucleotide. The term “UMI” is used herein to refer to both the sequence information of a polynucleotide and the physical polynucleotide per se. A UMI can range in length from about 2 to 100 nucleotide bases or more, and preferably has a length between about 4-16 nucleotide bases. The UMI can be a consecutive sequence or may be split into several subunits. Each of these subunits may be present in separate oligonucleotides and/or adapters. These subunits are preferably used together to generate a substantially unique tag, preferably a fully unique tag, for a single polynucleotide. For instance, if a polynucleotide is a fragment flanked by two oligonucleotides, each of these two oligonucleotides may comprise a subunit of the UMI. In case the polynucleotide is a ligation product of two oligonucleotides, each of these two oligonucleotides may comprise a subunit of the UMI. In order to obtain a consensus sequence, the sequence reads obtained in the method of the invention may be grouped based on the information of each of the two UMI subunits. Preferably a UMI does not contain two or more consecutive identical bases. Furthermore, there is preferably a difference between UMIs of at least two, preferably at least three bases. A UMI may have random, pseudo-random or partially random, or a non-random nucleotide sequence. As a UMI can be used to uniquely identify the originating molecule from which the read is derived, reads of amplified polynucleotides can be collapsed into a single consensus sequence from each originating polynucleotide. A UMI may be fully or substantially unique. Fully unique is to be understood herein as that every polynucleotide provided in the method of the invention comprises a unique tag that differs from all the other tags comprised in further polynucleotides in the method of the invention. Substantially unique is to be understood herein in that each polynucleotide provided in the method, product, composition or kit of the invention comprises a random UMI, but a low percentage of these polynucleotides may comprise the same UMI. Preferably, substantially unique molecular identifiers are used in case the chances of tagging the exact same molecule comprising the sequence of interest with the same UMI is negligible. Preferably, a UMI is fully unique in relation to a specific sequence of interest. A UMI preferably has a sufficient length to ensure this uniqueness. In some implementations, a less unique molecular identifier (i.e. a substantially unique identifier, as indicated above) can be used in conjunction with other identification techniques to ensure that each DNA molecule is uniquely identified during the sequencing process. For instance, the UMI of the invention may be less unique such that different sequences of interest may be coupled to the same or similar UMI. In the latter case, the combination of the sequence information of the UMI together with the sequence information of the sequence of interest allows for the identification of the originating polynucleotide. A UMI is preferably used to determine that all reads from a single cluster are identified as deriving from a single molecule.

A UMI can be considered as a specific type of barcode that serves to identify a specific nucleic acid molecule. Further barcodes may serve to identify e.g. a type of target fragment and/or a sample. Like a UMI, a barcode can be considered as a stretch of a defined number and sequence of nucleotides with similar structural features as indicated herein for a UMI. In case a barcode is a sample barcode, each barcoded nucleic acid molecule or target fragment of a sample may comprise the same barcode. In case a barcode is a target fragment barcode, optionally each specific type of target fragment that may be present in a multitude of different samples may be barcoded with the same target fragment barcode, while within each sample different target fragments may be barcoded with different target fragment barcodes. Such target fragment barcode allows for the easy clustering of sequence data for instance after processing samples by a method such as described herein and subsequently sequencing. In order to allow for de-multiplexing to allocate sequences of specific target fragments to their originating samples, barcoded target fragments are preferably barcoded with both a sample barcode and a target fragment barcode.

### DETAILED DESCRIPTION

The inventors discovered a versatile method for the labelling of a target nucleic acid fragment, wherein the target nucleic acid fragment may comprise a sequence of interest. More in particular, in the method of the invention, a target nucleic acid molecule is labelled on one or both sides with a specific nucleotide sequence. This newly added nucleotide sequence can subsequently be used in further downstream processes, e.g. to anneal primers to the specifically added sequence, or to couple additional sequences to the target nucleic acid fragment, such as adapter sequences for deep-sequencing. Coupling the adapter sequences to only the target nucleic acid fragments results in selective sequencing of the target nucleic acid fragments. Similarly, annealing a protective adapter to the labelled nucleic acid fragment and subsequent exonuclease protection results in the enrichment of the target nucleic acid fragment in a sample. The method as detailed herein below can therefore also be at least one of:
i) a method for the enrichment of a target nucleic acid fragment;
ii) a method for extending a target nucleic acid fragment;
iii) a method for library preparation;
iv) a method of sequencing, preferably bi-directional sequencing and/or combinatorial barcode sequencing; and
v) a method for amplifying, preferably selectively amplifying, a target nucleic acid fragment.

In a first aspect, the invention pertains to a method for labelling a target nucleic acid fragment, wherein the target nucleic acid fragment comprises a first strand and a complementary second strand. Preferably, the target nucleic acid fragment comprises a sequence of interest. The method preferably comprises the steps of:
a) providing a sample comprising a double-stranded nucleic acid molecule, wherein the double-stranded nucleic acid molecule comprises the sequence of interest;
b) contacting the double-stranded nucleic acid molecule with a site-specific nuclease to generate a double-stranded break, wherein the double-stranded break results in a free 3′-end of the first strand of the target nucleic acid fragment; and
c) contacting the cleaved double-stranded nucleic acid molecule with a DNA polymerase and a template molecule, preferably a reverse transcriptase and a template RNA molecule, thereby labelling the free 3′-end of the first strand of the target nucleic acid fragment with one or more nucleotides

Optionally, the site-specific nuclease in step b) and the reverse transcriptase in step c) are separate 40 entities. Exemplary embodiments are schematically depicted in FIG. 1.

The method of the invention can be an in vitro method.

Optionally, the method of the invention results in the parallel or subsequent labelling of multiple target nucleic acid fragments. Preferably, the method of the invention comprises the labelling of multiple target nucleic acid fragments from one or more nucleic acid samples. Such method may be considered a method for preparing a nucleic acid library for downstream processing, such as sequencing.

The term “labelling” in context of the invention is to be understood as the addition of one or more nucleotides to a target nucleic acid fragment. These newly added nucleotides are preferably added in a predetermined sequence. This sequence is preferably complementary to a part of the sequence of the template RNA molecule as defined herein. The sequence of the label is preferably complementary to a sequence that is located at the 5′ end of the template RNA molecule. Preferably, the method of the invention can add at least one nucleotide to at least one end of the target nucleic acid fragment. Preferably, the method of the invention can add at least about 1, 2, 5, 10, 15, 20, 25, 30 or more nucleotides to at least one or both ends of the target nucleic acid fragment. Optionally, the method of the invention can add about 10-150, 11-100, 12-90, 13-14-70, 15-60, 16-50, 17-25, 18-150, 19-100, 20-90, 21-80, 22-70, 23-60 or about 24-50 nucleotides to at least one or both ends of the target nucleic acid fragment.

### Providing a sample—Step a)

In step a) of the method of the invention a sample is provided, wherein the sample comprises a double-stranded nucleic acid molecule. The double-stranded DNA molecule preferably comprises the target nucleic acid fragment, which target nucleic acid fragment preferably comprises a sequence of interest. Preferably, the double-stranded nucleic acid molecule thus comprises the sequence of interest.

The nucleic acid sample of the method of the invention may be from any source, e.g. human, animal, plant, microorganism, bacterium, virus, and may be of any kind, e.g. endogenous or exogenous to the cell, for example genomic DNA, chromosomal DNA, artificial chromosomes, plasmid DNA, or episomal DNA, cDNA, RNA, mitochondrial, or of an artificial library such as a BAC or YAC or the like. The DNA may be nuclear or organellar DNA. Preferably, the DNA is chromosomal DNA, preferably endogenous to the cell.

The double-stranded nucleic acid of step a) may be isolated and/or purified, preferably from a biological source. Optionally, the double-stranded nucleic acid of step a) is synthetic. Optionally, the double-stranded nucleic acid of step a) is synthetic DNA, optionally single- or double-stranded DNA reverse-transcribed from RNA.

The double-stranded nucleic acid molecule of step a) may originate from a virus or a living organism, such as a living human, animal or plant. Optionally, said double-stranded nucleic acid is isolated and/or purified from the virus or living organism. The, optionally isolated and/or purified, nucleic acid from a virus or living organism may subsequently be amplified and/or reverse transcribed, resulting in synthetic DNA. The sample of step a) may originate from a single cell, a collection of single cells, (part of) a tissue, (part of) an organ and/or a fluid. The double-stranded nucleic acid isolated from a cell may be obtained by a method comprising a step of lysing the cell. The double-stranded nucleic acid molecule of step a) may therefore be a double-stranded nucleic acid molecule of a lysed cell. The double-stranded nucleic acid molecule of step a) may be an extracellular double-stranded nucleic acid.

Preferably, in case the sample is a of human or animal origin, said sample is obtained by a non-invasive or minimal invasive method.

It is understood herein that the nucleic acid sample comprises at least one target nucleic acid fragment. Put differently, the nucleic acid sample thus may comprise 1, 2, 3, 4, 5, 6, 7, 8, 9, 10 or more target nucleic acid fragments, such as at least about 50, 100, 150, 200, 250, 300, 350, 400, 450, 500, 750, 1000 or more target nucleic acid fragments, wherein preferably each target nucleic acid fragment within the sample has a distinct sequence of interest.

It is further understood herein that a single double-stranded nucleic acid molecule within a sample comprises at least one target nucleic acid fragment, wherein the at least one target nucleic acid fragment comprises a sequence of interest. Put differently, a single double-stranded nucleic acid molecule may comprise 1, 2, 3, 4, 5, 6, 7, 8, 9, 10 or more target nucleic acid fragments, such as at least about 50, 100, 150, 200, 250, 300, 350, 400, 450, 500, 750, 1000 or more target nucleic acid fragments, wherein preferably each target nucleic acid fragment within the double-stranded nucleic acid molecule has a distinct sequence of interest.

### Cleaving the Double-Stranded DNA Molecule—step b) and step d)

In step b) of the method of the invention, the double-stranded nucleic acid molecule is contacted with a site-specific nuclease to generate a double-stranded break. Thus the double-stranded break is generated at a specific location. Preferably the double-stranded break is generated at a location that is in close vicinity to the sequence of interest. Preferably, the generated double-stranded break is located immediately next to the sequence of interest. The double-stranded break may be generated upstream or downstream of the sequence of interest and can result in the free 3′ or 5′ end of the target nucleic acid fragment. The double-stranded break generates a free 3′-end of the first strand of the target nucleic acid fragment. It is understood herein that this free 3′-end of the first strand of the target nucleic acid fragment can be the free 3′-end of the top or bottom strand of the target nucleic acid fragment. The site-specific nuclease may be designed such that it remains bound to the part of the cleaved nucleic acid molecule that comprises the sequence of interest at least throughout the subsequent labelling step as further defined herein. Preferably, the site-specific nuclease is designed such that it remains bound to the target nucleic acid fragment at least throughout step c). Preferably, the site-specific nuclease is designed such that it is remains located at the site to be labelled.

Simultaneously with step b) or after step b), the double-stranded nucleic acid molecule can be contacted with a second site-specific nuclease to generate a second double-stranded break. The method of the invention thus may comprise a step d) wherein the double-stranded nucleic acid molecule is contacted with a second site-specific nuclease to generate a second double-stranded break. Preferably, the second double-stranded break results in a free 3′-end of the second strand of the target nucleic acid fragment.

Preferably, step d) is performed simultaneously with step b). Step d) may be performed after step b), and before step c). Alternatively or in addition, step d) may be performed after step c).

Preferably this second double-stranded break is generated at a location that is in close vicinity to the sequence of interest. Preferably, the second generated double-stranded break is located immediately next to the sequence of interest. The double-stranded break may be generated upstream or downstream of the sequence of interest and can result in the free 3′ or 5′ end of the target nucleic acid fragment. The double-stranded break generates a free 3′-end of the second strand of the target nucleic acid fragment. It is understood herein that this free 3′-end of the second strand of the target nucleic acid fragment can be a free 3′-end of the top or bottom strand of the target nucleic acid fragment.

In case two double-stranded breaks are generated, the first double-stranded break may generate the 3′ end of the first strand of the target nucleic acid fragment and the second double-stranded break may generate the 5′ end of the first strand of the target nucleic acid fragment. In case two double-stranded breaks are generated, the first double-stranded break may generate the end of the second strand of the target nucleic acid fragment and the second double-stranded break may generate the 3′ end of the second strand of the target nucleic acid fragment.

The cleavage step b), and optionally cleavage step d), is preferably performed under experimental conditions wherein the site-specific nuclease is capable of specifically binding and cleaving the double-stranded nucleic acid molecule, i.e. under experimental conditions wherein the site-specific nuclease shows specific enzymatic activity. Such experimental conditions are well-known by the skilled person and/or can be determined using any conventional means. These experimental conditions may be dependent on the type of site-specific nuclease, as will be known to the skilled person. The experimental conditions can be the same or similar as the conditions described in the experimental section below.

It is understood herein that the sequence of interest is present in the double-stranded nucleic acid molecule prior to cleavage with the site-specific nuclease(s). Cleavage of the nucleic acid molecule results in at least two or more nucleic acid fragments, wherein at least one nucleic acid fragment is a target nucleic acid fragment. The other generated nucleic acid fragment can also be, or may comprise, a target nucleic acid fragment or is a non-target nucleic acid fragment. The target nucleic acid fragment comprises or consists of the sequence of interest. Hence, prior to cleaving the double-stranded nucleic acid molecule, it is clear for the skilled person that the target nucleic acid fragment is encompassed within the double-stranded nucleic acid molecule and the target nucleic acid fragment is released from the double-stranded nucleic acid molecule upon cleavage with at least one site-specific endonuclease.

The site-specific nuclease generating the first, and optional second, double-stranded break can be selected from the group consisting of a CRISPR-nuclease complex, a nucleic acid-Argonaute complex, Zinc finger nucleases, TALENs and meganucleases. Preferably, the site-specific nuclease in step b) and/or step d) is a CRISPR-nuclease complex.

### CRISPR-Nuclease Complex

The CRISPR-nuclease complex, or complexes, for use according to the invention are to be understood herein as a CRISPR associated (CAS) protein, or CRISPR-nuclease, complexed with a guide RNA.

A CRISPR-nuclease comprises a nuclease domain and at least one domain that interacts with a guide RNA. When complexed with a guide RNA, the CRISPR-nuclease is directed to a specific nucleic acid sequence by a guide RNA. The guide RNA interacts with the CRISPR-nuclease as well as with the specific target nucleic acid sequence, such that, once directed to the site comprising the specific nucleic acid sequence via the guide sequence, the CRISPR-nuclease is able to introduce a break at the target site. Preferably, the CRISPR-nuclease is able to introduce a single or double strand break at the target site, in case one or both domains of the nuclease are catalytically active, respectively. The skilled person is well aware of how to design a guide RNA in a manner that it, when combined with a CRISPR-nuclease, effects the introduction of a single- or double-stranded break at a predefined site in the nucleic acid molecule. Preferably, the CRISPR-nuclease effects the introduction of a double-stranded beak.

CRISPR-nucleases can generally be categorized into six major types (Type I-VI), which are further subdivided into subtypes, based on core element content and sequences (Makarova et al, 2011, Nat Rev Microbiol 9:467-77 and Wright et al, 2016, Cell 164(1-2):29-44). In general, the two key elements of a CRISPR-CAS system complex are a CRISPR-nuclease and a guide RNA.

Type II CRISPR-CAS systems include a signature Cas9 protein, a single protein (about 160 KDa), capable of specifically cleaving duplex DNA. The Cas9 protein typically contains two nuclease domains, a RuvC-like nuclease domain near the amino terminus and the HNH (or McrA-like) nuclease domain near the middle of the protein. Each nuclease domain of the Cas9 protein is specialized for cutting one strand of the double helix (Jinek et al, 2012, Science 337 (6096): 816-821). The Cas9 protein is an example of a CAS protein of the type II CRISPR/-CAS system and forms a CRISPR-nuclease complex, when combined with the crRNA and a second RNA termed the trans-activating crRNA (tracrRNA). The crRNA and tracrRNA function together as the guide RNA. The CRISPR-nuclease complex introduces DNA double strand breaks (DSBs) at the position in the genome defined by the crRNA. Jinek et al. (2012, Science 337: 816-820) demonstrated that a single chain chimeric guide RNA (“sgRNA”) produced by fusing an essential portion of the crRNA and tracrRNA was able to form a functional CRISPR-nuclease complex in combination with the Cas9 protein.

The Type V CRISPR-CAS system includes the Clustered Regularly Interspaced Short Palindromic Repeats from Prevotella and Francisella 1 or CRISPR/Cpf1. Cpf1 genes are associated with the CRISPR locus and code for an endonuclease that use a crRNA to target DNA. Cpf1 is a smaller and simpler endonuclease than Cas9. Cpf1 is a single RNA-guided endonuclease lacking a tracrRNA, and it preferably utilizes a T-rich protospacer-adjacent motif. Cpf1 cleaves DNA via a staggered DNA double-stranded break (Zetsche et al (2015) Cell 163 (3): 759-771). The type V CRISPR-CAS system preferably includes at least one of Cpf1, C2c1 and C2c3.

The CRISPR-nuclease complex, or complexes, for use in the method of the invention may comprise any CRISPR-nuclease capable of generating a double-stranded break. Preferably, the CRISPR-nuclease complex, or complexes, for use in the method of the invention comprises a Type II CRISPR-nuclease, e.g., Cas9 (e.g., the protein of SEQ ID NO: 1, encoded by SEQ ID NO: 2, or the protein of SEQ ID NO: 3) or a Type V CRISPR-nuclease, e.g. Cpf1 (e.g., the protein of SEQ ID NO: 4, encoded by SEQ ID NO: 5) or Mad7 (e.g. the protein of SEQ ID NO: 6 or 7), or a protein derived thereof, having preferably at least about 70%, 80%, 90%, 91%, 92%, 93%, 94%, 95%, 96%, 97%, 98%, or 99% sequence identity to said protein over its whole length. Preferably CRISPR-nuclease complex, or complexes, for use the method of the invention comprises a Type II CRISPR-nuclease, preferably a Cas9 nuclease.

The skilled person knows how to prepare the different components of the CRISPR-nuclease complex. In the prior art, numerous reports are available on its design and use. See for example the review by Haeussler et al (J Genet Genomics. (2016)43(5):239-50. doi: 10.1016/j.jgg.2016.04.008.) on the design of guide RNAs and its combined use with a CAS-protein (originally obtained from S. pyogenes), or the review by Lee et al. (Plant Biotechnology Journal (2016) 14(2) 448-462).

Preferably, the CRISPR-nuclease, such as Cas9, comprises two catalytically active nuclease domains. For example, a Cas9 protein can comprise a RuvC-like nuclease domain and an HNH-like nuclease domain. The RuvC and HNH domains work together, both cutting a single strand, to make a double-stranded break in DNA. (Jinek et al., Science, 337: 816-821).

A dead CRISPR-nuclease comprises modifications such that none of the nuclease domains shows cleavage activity. The CRISPR-nuclease for use in the method of the invention may be a variant of a CRISPR-nuclease wherein one of the nuclease domains is mutated such that it is no longer functional (i.e., the nuclease activity is absent), thereby creating a nickase. An example is a SpCas9 variant having either the D10A or H840A mutation. Preferably, the nuclease of the CRISPR-nuclease complex is not a dead nuclease. Preferably, the CRISPR-nuclease of the CRISPR-nuclease complex, or complexes, is either a nickase or (endo)nuclease, preferably an (endo)nuclease. The CRISPR-nuclease complex, or complexes, used in the method of the invention may comprise a whole Cas9 protein or may comprise a functional fragment thereof.

Preferably the CRISPR-nuclease comprises a Cas9 or Cpf1 nuclease, preferably a Cas9 nuclease. Preferably, CRISPR-nuclease complex, or complexes, for use in the invention comprises a Cas9 protein. The Cas9 protein may be derived from the bacteria Streptococcus pyogenes (SpCas9; NCBI Reference Sequence NC_017053.1; UniProtKB-Q99ZW2), Geobacillus thermodenitrificans (UniProtKB-A0A178TEJ9), Corynebacterium ulcerous (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychrotlexus torquisl (NCBI Ref: NC_018721.1); Streptococcus thermophilus (NCBI Ref: YP_820832.1); Listeria innocua (NCBI Ref: NP_472073.1); Campylobacter jejuni (NCBI Ref: YP_002344900.1); or Neisseria meningitidis (NCBI Ref: YP_002342100.1). Encompassed are Cas9 variants from these, having an inactivated HNH or RuvC domain homologues to SpCas9, e.g. the SpCas9_D10A or SpCas9_H840A, or a Cas9 having equivalent substitutions at positions corresponding to D10 or H840 in the SpCas9 protein, rendering a nickase. Preferably, the Cas9 protein for use in the method of the invention is an (endo)nuclease.

The programmable nuclease may be derived from Cpf1, e.g., Cpf1 from Acidaminococcus sp; UniProtKB-U2UMQ6. The variant may be a Cpf1-nickase having an inactivated RuvC or NUC domain, wherein the RuvC or NUC domain has no nuclease activity anymore. The skilled person is well aware of techniques available in the art such as site-directed mutagenesis, PCR-mediated mutagenesis, and total gene synthesis that allow for inactivated nucleases such as inactivated RuvC or NUC domains. An example of a Cpf1 nickase with an inactive NUC domain is Cpf1 R1226A (see Gao et al. Cell Research (2016) 26:901-913, Yamano et al. Cell (2016) 165(4): 949-962). In this variant, there is an arginine to alanine (R1226A) conversion in the NUC-domain, which inactivates the NUC-domain. Preferably the Cpf1 protein is not an inactivated Cpf1 protein. Preferably, the Cpf1 protein for use in the invention is an (endo)nuclease.

The method of the invention may provide for a simultaneous enrichment of these target nucleic acid fragments from a nucleic acid sample. Therefore optionally, in step b) of the method of the invention, multiple CRISPR-nuclease complexes are added for enrichment, isolation or sequencing of multiple target nucleic acid fragments from a nucleic acid sample. Preferably, these multiple CRIRPR-nuclease complexes may comprise the same CRISPR-nuclease, but may differ in their guide RNA. For example, for each target nucleic acid fragment, two distinct guide RNA molecules may be used, e.g. one guide RNA is incorporated in the first CRISPR-nuclease complex another guide RNA is incorporated in the second CRISPR-nuclease complex. For e.g. at least about 100, 150, 200, 250, 300, 350, 400, 450, 500, 750, 1000 or more target nucleic acid fragments, preferably at least about 50, 100, 150, 200, 250, 300, 350, 400, 450, 500, 750, 1000 or more sets of guide RNA molecules, preferably at least about 100, 200, 300, 400, 500, 600, 700, 800, 900, 1000, 1500, 2000 or more different guide RNA molecules may be used in the method of the invention.

### Guide RNA

The CRISPR-nuclease complex, or complexes, for use in the method of the invention further comprise a CRISPR-nuclease associated guide RNA that directs the complex to a defined target site in the double-stranded nucleic acid molecule, also named the protospacer sequence. A guide RNA comprises a guide sequence for targeting the CRISPR-nuclease complex to the protospacer sequence that is preferably near, at or within the sequence of interest in the double-stranded nucleic acid molecule, and may be a sgRNA or the combination of a crRNA and a tracrRNA (e.g. for Cas9) or a crRNA only (e.g. in case of Cpf1). The CRISPR-nuclease complex for use in the method of the invention may thus comprise a guide RNA, wherein the guide RNA is a combination of a crRNA and a tracrRNA, and wherein preferably the (endo)nuclease is Cas9. The crRNA and tracrRNA are optionally combined into a sgRNA (single guide RNA). Alternatively, the CRISPR-nuclease complex for use in the method of the invention may comprise a guide RNA, wherein the guide RNA is a crRNA, and wherein preferably the (endo)nuclease is Cpf1.

The term “guide RNA” is thus understood herein to refer to the RNA molecule, or combination of RNA molecules that direct the (endo)nuclease to specific nucleotide sequence within the double-stranded DNA molecule. In case of the Cas9 (endo)nuclease, the term “guide RNA” thus encompasses both the combination of a crRNA and an tracrRNA, as well as a single guide RNA (sgRNA), except if it is clear from the context that only the combination of a crRNA and an tracrRNA, or only a single guide RNA is intended. In case of the Cpf1 (endo)nuclease, the term “guide RNA” refers to the crRNA.

Optionally, more than one type of guide RNA may be used in the same method, for example aimed at two or more different sequences of interest, or aimed at two different locations of the same sequence of interest, for example aimed at a sequence upstream and a sequence downstream of the same sequence of interest. As a non-limiting example, a first guide RNA may guide a first CRISPR-nuclease complex to a sequence in the double-stranded nucleic acid, such that the nucleic acid molecule is cleaved upstream of the sequence of interest, and a second guide RNA may guide a second CRISPR-nuclease complex to another sequence in the double-stranded nucleic acid, such that the nucleic acid molecule is cleaved downstream of the sequence of interest.

Preferably, the CRISPR-nuclease complex comprises a CRISPR-nuclease that cleaves the nucleic acid within the protospacer sequence. A preferred CRISPR-nuclease is Cas9.

Molecules suitable as crRNA and tracrRNA for use as gRNA (guide RNA) in a CRISPR-nuclease complex are well known in the art (see e.g., WO2013142578 and Jinek et al., Science (2012) 337, 816-821).

At least one of the guide RNAs for use in the method of the invention may comprise a sequence that can hybridize to or near a sequence of interest, preferably a sequence of interest as defined herein. Preferably, at least one of the guide RNAs comprises a nucleotide sequence that is fully complementary to a sequence in the sequence of interest i.e. the sequence of interest comprises a protospacer sequence.

Alternatively or in addition, at least one of the guide RNAs for use in the method of the invention may comprise a sequence that can hybridize to or near the complement of a sequence of interest, preferably a sequence of interest as defined herein. Preferably, at least one of the guide RNAs comprises a nucleotide sequence that has full sequence identity with, or with a part of, the sequence of interest.

The part of the crRNA sequence that is complementary to the protospacer sequence is designed to have sufficient complementarity with the protospacer sequence to hybridize with the protospacer sequence and direct sequence-specific binding of a complexed nuclease. The protospacer sequence is preferably adjacent to a protospacer adjacent motif (PAM) sequence, which PAM sequence may interact with the CRISPR nuclease of the RNA-guided CRISPR-system nuclease complex as defined herein. For instance, in case the CRISPR nuclease is S. pyogenesCas9, the PAM sequence preferably is 5′-NGG-3′, wherein N can be any one of T, G, A or C. The skilled person is capable of engineering the crRNA to target any desired sequence, preferably by engineering the sequence to be at least partly complementary to any desired protospacer sequence, in order to hybridize thereto. Preferably, the complementarity between part of a crRNA sequence and its corresponding protospacer sequence, when optimally aligned using a suitable alignment algorithm, is at least about 70%, 80%, 85%, 90%, 91%, 92%, 93%, 94%, 95%, 96%, 97%, 98% or 100%. The part of the crRNA sequence that is complementary to the protospacer sequence may be at least about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some preferred embodiments, a sequence complementary to the DNA target sequence is less than about 75, 50, 45, 40, 35, 30, 25, 20 nucleotides in length. Preferably, the length of the sequence complementary to the DNA sequence is at least 17 nucleotides. Preferably the complementary crRNA sequence is about 10-30 nucleotides in length, about 17-25 nucleotides in length or about 15-21 nucleotides in length. Preferably the part of the crRNA that is complementary to the protospacer sequence is 15, 16, 17, 18, 19, 20, 21, 22, 23, 24 or 25 nucleotides in length, preferably 20 or 21 nucleotides, preferably 20 nucleotides.

Preferably in the embodiment wherein the crRNA and tracrRNA are separate molecules and wherein the method comprises a step b) and a step d) to generate two double-stranded breaks in the nucleic acid molecule, the first and second CRISPR-nuclease complexes may comprise a first and a second crRNA, respectively, wherein the first and second crRNA do not have an identical sequence. Preferably, the first and second crRNA recognize a different protospacer sequence. The first and second CRISPR-nuclease complexes however may comprise tracrRNAs having identical or nearly identical sequences.

Preferably, the crRNA and tracrRNA are linked to together to form a sgRNA. The crRNA and tracrRNA can be linked, preferably covalently linked, using any conventional method known in the art. Covalent linkage of the crRNA and tracrRNA is e.g. described in Jinek et al. (supra) and WO13/176772, which are incorporated herein by reference. The crRNA and tracrRNA can be covalently linked using e.g. linker nucleotides or via direct covalent linkage of the 3′ end of the crRNA and the 5′ end of the tracrRNA.

Preferably, the guide RNA of the CRISPR nuclease complex, or complexes, is designed such that upon incubation of the nucleic acid sample with the CRISPR-nuclease complex, or complexes, the target nucleic acid fragment comprised within a nucleic acid molecule from the nucleic acid sample is excised from said nucleic acid molecule. In addition, preferably the first guide RNA is designed such that the first CRISPR-nuclease complex remains bound to the target nucleic acid fragment after cleavage of the nucleic acid molecule. In addition preferably the optional second guide RNA is designed such that the second CRISPR-nuclease complex remains bound to the target nucleic acid fragment after the second cleavage of the nucleic acid molecule.

The target nucleic acid fragment when present in the double-stranded nucleic acid molecule can be flanked by at least one non-target nucleic acid fragment. The target nucleic acid fragment when present in the double-stranded nucleic acid molecule may be flanked on both sides with a non-target nucleic acid fragment, i.e. one non-target nucleic acid fragment may be present directly upstream of the target nucleic acid fragment and one non-target nucleic acid fragment may be present directly downstream of the target nucleic acid fragment.

Step b) and d) of the method of the invention may be performed by incubating the CRISPR-nuclease complex, or complexes, and the nucleic acid sample together at conditions and time suitable for the CRISPR-nuclease complex, or complexes, to induce a double strand break, such as, but not limited to, the conditions detailed in the Examples provided herein. Optionally, the incubation is performed between about 1 min to about 18 hours, preferably about 60 minutes, at about 10-90° C., preferably about 37° C.

In case the site-specific nuclease generating a double-stranded break is an Argonaute, the term “guide RNA” as detailed herein may be replaced for a guide nucleic acid, wherein the guide nucleic acid is preferably at least one of a small RNA or a small DNA guide. The nucleic acid Argonaute complex is thus preferably a guide nucleic acid—Argonaute complex, preferably at least one of an guide RNA—Argonaute complex and a guide DNA—Argonaute complex.

### Labelling the free 3′-End of the Target Nucleic Acid Rragment—Step c) and e)

Cleaving the double-stranded nucleic acid molecule generates a free 3′-end of the target nucleic acid fragment. This free 3′-end can subsequently be labelled or “extended” with one or more nucleotides, preferably the nucleotides extending the 3′-end of the target nucleic acid fragment have a predetermined sequence. The step of labelling of the 3′-end of the target nucleic acid fragment with one or more nucleotides is preferably performed by contacting the cleaved double-stranded nucleic acid molecule with a reverse transcriptase and a template RNA molecule. The reverse transcriptase uses the template RNA as a template for extending the free 3′ end of the nucleic acid fragment, thereby adding to the 3′-end one or more nucleotides that are complementary to the template RNA molecule. Put differently, the reverse transcriptase thus reversely transcribes part of the template RNA.

The method may comprise a step e) of contacting the target nucleic acid fragment with a DNA polymerase and a second template molecule, preferably with a reverse transcriptase and a second template RNA molecule, thereby labelling the second strand of the target nucleic acid fragment at the free 3′-end with one or more nucleotides, wherein preferably step e) is performed simultaneously with step c).

Preferably, the second site-specific nuclease of step d) may be designed such that it remains bound to the part of the cleaved nucleic acid molecule that comprises the sequence of interest at least throughout the subsequent labelling step as further defined herein. Preferably, the site-specific nuclease of step d) is designed such that it remains bound to the target nucleic acid fragment at least throughout step e). Preferably, the site-specific nuclease is designed such that it is remains located at the site to be labelled.

The labelling step c), and optionally step e), is preferably performed under experimental conditions wherein the reverse transcriptase is capable of reversely transcribing the template RNA molecule, i.e. under experimental conditions wherein the reverse transcriptase shows enzymatic activity. Such experimental conditions are well-known by the skilled person and/or can be determined using any conventional means. These experimental conditions may be dependent on the type of Reverse Transcriptase, as will be known to the skilled person. The experimental conditions can be the same or similar as the conditions described in the experimental section below. These experimental conditions preferably at least include the presence of nucleotides, preferably naturally occurring nucleotides, preferably these experimental conditions include the presence of dNTPs, preferably at least one of adenine, guanine, cytosine and thymidine and optionally uracil.

The method of the invention thus may comprise a step c) of contacting the cleaved target nucleic acid molecule with a reverse transcriptase and a template RNA molecule, thereby labelling the free 3′-end of the first strand of the target nucleic acid fragment with one or more nucleotides. Simultaneously with step c) or after step c), the method may further comprise a step e) of contacting the target nucleic acid fragment with a DNA polymerase and a second template molecule, preferably a reverse transcriptase and a second template RNA molecule, thereby labelling the second strand of the target nucleic acid fragment at the free 3′-end with one or more nucleotides. Preferably, said step e) is performed simultaneously with step c). By labelling both the first and second strand, a double stranded fragment with two labels at either side of the target nucleic acid fragment is obtained.

Step c) is preferably performed after step a) and after step b). Optionally step c) is performed after step d). Step e) is preferably performed after step d). Thus in the method of the invention, the double-stranded nucleic acid molecule may first be cleaved all desired (e.g. one or more) locations, followed by contacting cleaved molecule with the RNA template molecules and a reverse transcriptase. Alternatively, the cleavage step and labelling step may be performed in an alternating fashion.

The method of the invention may thus comprise the following order of steps:
Steps a), b) and c)
Steps a), b), c), and d)
Steps a), b), c), d) and e)
Steps a), b), d) c) and e)

The contacting of steps b) and step c) may occur sequentially and simultaneously. In other words, the reaction components of step b) and c) may be added to the reaction mixture sequentially and simultaneously, however as the site-specific nuclease of step b) may serve to make the free 3′ end accessible for the template RNA of step c) to bind, the site-specific nuclease should preferably remain to be present and bound to the target fragment throughout step c) of the method of the invention. Optional step d) may be performed separately at a later stage or simultaneously with steps b) and c).

In case of labelling of the target fragment at both sides, the method of the invention comprises step d) and e). The contacting of steps d) and step e) may occur sequentially and simultaneously. In other words, the reaction components of step d) and e) may be added to the reaction mixture sequentially and simultaneously, however as the site-specific nuclease of step d) may serve to make the free 3′ end accessible for the template RNA of step e) to bind, said complex should preferably remain to be present and bound to the target fragment throughout step e) of the method of the invention.

In a preferred embodiment, and in case of labelling of the target fragment at both sides, the reaction components of steps b), c), d) and e) may all be added to the reaction mixture simultaneously.

In case the generation of the double-stranded break and the labelling is performed simultaneously preferably the experimental conditions within said reaction vessel is such that it allows for both the cleaving by the site-specific nuclease and the labelling by the DNA polymerase.

As detailed herein, the invention may further comprise at least one of steps f) and g) and/or may further comprise at least one of steps (i), (ii) and (iii).

Preferably, the free 3′-end of the first strand and the free 3′-end of the second strand of the target nucleic acid fragment is extended by the addition of one or more nucleotides. The sequence of the one or more nucleotides extending the first strand can be identical or nearly identical to the sequence of the nucleotides extending the second strand of the target nucleic acid fragment. The one or more nucleotides extending the first and second strand may have more than 20%, 30%, 40%, 50%, 60%, 70%, 80%, 90%, 95% or 100% nucleotide sequence identity.

Preferably, the sequence of the one or more nucleotides extending the first strand is different from the sequence of the nucleotides extending the second strand of the target nucleic acid fragment. Preferably, the one or more nucleotides extending the first and second strand have less than 98%, 95%, 90%, 85%, 80%, 75%, 70%, 65%, 60%, 55%, 50%, 45%, 40%, 35%, 30%, 25%, 20%, 15% or even less than 10% nucleotide sequence identity.

The number of nucleotides extending the free 3′-end of the first strand can be identical to the number of nucleotides extending the free 3′-end of the second strand of the target nucleic acid fragment. Alternatively, the number of nucleotides extending the free 3′-end of the first strand differs from the number of nucleotides extending the free 3′-end of the second strand of the target nucleic acid fragment. The number of nucleotides extending the first stand and the number of nucleotides extending he second strand may differ by at least about 1, 2, 4, 6, 8, 10, 20 or more nucleotides.

The sequence of the one or more nucleotides extending the first and/or second strand of the target nucleic acid fragment may comprise a functional domain, preferably selected from the group consisting of a restriction site domain, a capture domain, a sequencing primer binding site, an amplification primer binding site, a detection domain, a barcode sequence, a transcription promoter domain and a PAM sequence, or any combination thereof. The barcode can be, but is not limited to, a sample barcode, an allele specific identifier, a locus specific identifier or a unique molecular identifier (UMI). In case for instance two allelic variants of a specific target nucleic acid may be present in the sample, and the template RNA molecule is capable discriminating between one or more polymorphisms by annealing to only one of them, any barcode within this RNA molecule may serve as allele specific identifier.

The method of the invention may comprise a step e) as defined herein, wherein the resulting target nucleic acid fragment is labeled at either side of the target nucleic acid fragment. The label at the 3′ end of the first strand (the first label) may comprise a functional domain. Alternatively or in addition, the label at the 3′ end of the second strand (the second label) may comprise a functional domain. The functional domain located in the first and second label may be the same functional domain or different functional domains. For instance, the label at the 3′ end of the first strand of the target nucleic acid fragment may comprise a first primer binding site, and the label at the 3′ end of the second strand of said target nucleic acid fragment may comprise a second primer binding site.

Said first and second primer binding site may comprise a sequence for annealing a first and second amplification primer, respectively, and/or for annealing a first and second sequencing primer, respectively. Said first (amplification and/or sequencing) primer may be indicated as a reverse primer, and said second (amplification and/or sequencing) primer may be indicated as a forward primer. At least one, or preferably both of the strands of the resulting double labelled nucleic acid fragment may be used as template molecule for amplification and/or sequencing. For instance the first and second labelled strand may be used for bi-directional sequencing.

In addition or alternatively, the label at the 3′ end of the first strand of the target nucleic acid fragment may comprise a first barcode, and the label at the second strand of said target nucleic acid fragment may comprise a second barcode. The first and/or second barcode optionally is a first and/or a second UMI. These two barcodes together may form a combinatorial barcode or combinatorial sequence barcode, e.g. as described in WO2011/155833, which is incorporated herein by reference. In brief, the combined sequences of these two barcodes (i.e. the combinatorial barcode or combinatorial sequence barcode) is used as an identifier. Optionally, the combined sequence of these two barcodes is used as a sample identifier. Optionally, the combined sequence of these two barcodes is used as an identifier of a specific target nucleic acid fragment.

Optionally, at least one of the first and second label comprises more than one barcode and/or more than one UMI.

Optionally, the label at the 3′ end of the first strand of the target nucleic acid fragment may comprise a first barcode and a first primer binding site, and the label at the second strand of said target nucleic acid fragment may comprise a second barcode and a second primer binding site. Preferably, the primer binding sites are sequencing primer binding sites. In case of a subsequent step of bi-directional sequencing, the first single-stranded template of the labelled nucleic acid fragment may comprise in a 5′ to 3′ direction a sequence of interest, a first barcode and a first sequencing primer binding site. The second single-stranded template of the labelled nucleic acid fragment may comprise in a 5′ to 3′ direction a sequence of interest, a second barcode and a second sequencing primer binding site. The first and second primer binding sites are located such that both the barcode and the sequence of interest of the resulting labelled fragment are sequenced form each single-stranded template using independent primer events, i.e. the reverse primer may be used for sequencing the first barcode and the sequence of interest of the first strand and the forward primer may be used for sequencing the second barcode and the sequence of interest of the second strand.

The label at the 3′ end of the first strand of the target nucleic acid fragment may comprise a first barcode and a first amplification primer binding site, and the label at the second strand of said target nucleic acid fragment may comprise a second barcode and a second amplification primer binding site. Optionally, the label may comprise a sequencing primer binding site. The first single-stranded template of the labelled nucleic acid fragment may comprise in a 5′ to 3′ direction a sequence of interest, a first barcode, an optional first sequencing primer binding site, and an amplification primer binding site. The optional second single-stranded template of the labelled nucleic acid fragment may comprise in a 5′ to 3′ direction a sequence of interest, a second barcode, an optional second sequencing primer binding site, and an amplification primer binding site. Preferably, the primer binding sites are located such that both the barcodes and the sequence of the target nucleic acid fragment are amplified using independent primer events, i.e. the reverse primer may be used to amplify the first barcode and the sequence of interest of the first strand and the forward primer may be used to amplify the second barcode and the (complementary) sequence of interest of the second strand.

In order to produce a labelled target nucleic acid fragment of the embodiments as indicated herein, the template RNA molecule of step c) and optionally step e) are specifically designed accordingly. In case the method of the invention is performed on multiple samples in a high throughput manner, preferably each target fragment of each sample is labelled with a label comprising a specific sample barcode such that for downstream processing, labelled target fragments from different samples can be pooled and processed together, while after sequencing the respective sequences can be allocated to its respective originating sample. In addition or alternatively to such sample barcode, the label may further comprise a UMI and/or a barcode for identification of a specific target fragment.

### Reverse Transcriptase (RT)

The protein labelling the free 3′end of the first and/or second strand may be any recombinant protein capable of extending the 3′-end of a double-stranded DNA molecule. Preferably, such protein is a DNA polymerase.

The polymerase may be wild type polymerases, functional fragment, mutants, variant, truncated variant, and the like. The polymerase may include a wild type polymerase from eukaryotic, prokaryotic, archael, or viral organism, and/or the polymerases may be modified by at least one of genetic engineering, mutagenesis and directed evolution-based processes.

The DNA polymerase may be a DNA-dependent and/or RNA-dependent DNA polymerase. The skilled person understands the invention is not limited to any particular RNA-dependent DNA polymerase or any particular DNA-dependent DNA polymerase. The term “RNA-dependent DNA polymerase” or “Reverse transcriptase” as defined herein may be replaced for the term “DNA-dependent DNA polymerase”, except it is clear from its context that the term “Reverse Transcriptase” is intended. Equally the term “template RNA molecule” may be replaced for a “template DNA molecule”, when used in conjunction with a DNA-dependent DNA polymerase.

Optionally in step c) and/or step e) a combination of 2, 3, 4 or more DNA polymerases can be used.

The polymerases are preferably “template-dependent” polymerases (i.e., a polymerase which synthesizes a nucleotide strand based on the order of nucleotide bases of a template strand).The DNA polymerase may be an DNA-dependent DNA polymerase. A preferred DNA-dependent DNA polymerase does not comprise strand replacement activity. A DNA polymerase that lacks strand replacement activity may label the 3′-end of the first and/or second strand, but is unable, or substantially unable, to elongate the provided template DNA molecule. The DNA-dependent DNA polymerase may naturally lack strand replacement activity or may be modified to lack strand replacement activity. A preferred DNA-dependent DNA polymerase that lack strand replacement activity is at least one of T4, T7 and Taq DNA polymerase.

The polymerase may include at least one of T7 DNA polymerase, T5 DNA polymerase, T4 DNA polymerase, Klenow fragment DNA polymerase, DNA polymerase III, and the like. The polymerase may be thermostable and/or and may include Taq, Tne, Tma, Pfu, Tfl, Tth, Stoffel fragment, VENT® and DEEPVENT® DNA polymerases, KOD, Tgo, JDE3, and mutants, variants and derivatives thereof (see e.g. U.S. Pat. Nos. 5,436,149; 4,889,818; 4,965,185; 5,079,352; 5,614,365; 5,374,553; 5,270,179; 5,047,342; 5,512,462; WO 92/06188; WO 92/06200; WO 96/10640; Barnes, W. M., Gene 112:29-35 (1992); Lawyer, E. C., et ah, PGR Meth. Appl. 2:275-287 (1993); Elaman, J.-M, et ah, Nuc. Acids Res. 22(15):3259-3260 (1994), each of which are incorporated by reference).

Optionally, the DNA polymerase lacks 3′ exonuclease activity. The DNA polymerase can be from bacteriophage. Bacteriophage DNA polymerases are generally devoid of 5′ to 3′ exonuclease activity, as this activity is encoded by a separate polypeptide. Examples of suitable DNA polymerases are T4, T7, and phi29 DNA polymerase.

Alternatively or in addition, the DNA polymerase is an archaeal polymerase. There are two different classes of DNA polymerases which have been identified in archaea: 1. Family B/pol I type (homologs of Pfu from Pyrococcus furiosus) and 2. pol II type (homologs of P. furiosus DP1/DP2 2-subunit polymerase). DNA polymerases from both classes have been shown to naturally lack an associated 5′ to 3′ exonuclease activity and to possess 3′ to 5′ exonuclease (proofreading) activity. Suitable DNA polymerases (pol I or pol II) can be derived from archaea with optimal growth temperatures that are similar to the desired assay temperatures.

A thermostable archaeal DNA polymerase can be isolated from Pyrococcus species (furiosus, species GB-D, woesii, abysii, horikoshii). Thermococcus species (kodakaraensis KODI, litoralis, species 9 degrees North-7, species JDE-3, gorgonarius), Pyrodictium occultum, and Archaeoglobus fulgidus.

The DNA Polymerase may be obtained from an eubacterial species. There are 3 classes of eubacterial DNA polymerases, pol I, II, and III. Enzymes in the Pol I DNA polymerase family possess 5′ to 3′ exonuclease activity, and certain members also exhibit 3′ to 5′ exonuclease activity. Pol II DNA polymerases naturally lack 5′ to 3′ exonuclease activity, but do exhibit 3′ to 5′ exonuclease activity. Pol III DNA polymerases represent the major replicative DNA polymerase of the cell and are composed of multiple subunits. The pol III catalytic subunit lacks 5′ to 3′ exonuclease activity, but in some cases 3′ to 5′ exonuclease activity is located in the same polypeptide.

There are a variety of commercially available Pol I DNA polymerases, some of which have been modified to reduce or abolish 5′ to 3′ exonuclease activity.

Suitable thermostable pol I DNA polymerases can be isolated from a variety of thermophilic eubacteria, including Thermus species and Thermotoga maritima such as Thermus aquaticus (Taq), Thermus thermophilus (Tth) and Thermotoga maritima (Tma UlTnna).

A preferred DNA-dependent DNA polymerase may be a prokaryotic or eukaryotic DNA-dependent DNA polymerase. A preferred prokaryotic DNA-dependent DNA polymerase is selected from the group consisting of Pol I, Pol II and Pol III. A preferred eukaryotic DNA-dependent DNA polymerase is selected from the group consisting of Pol a, Pol b, Pol g, Pol d, Pol e, and Pol z.

Preferably, the DNA polymerase is an RNA dependent DNA-polymerase or “Reverse Transcriptase”. The invention is not limited to any kind of specific reverse transcriptase (RT). In particular, the reverse transcriptase may be any naturally-occurring or recombinant protein capable of extending the 3′-end of a double-stranded DNA molecule. The reverse transcriptase preferably uses a template RNA to add a specific sequence of nucleotides to the 3′-end of the molecule, i.e. is an RNA-dependent DNA polymerase. The reverse transcriptase may be a naturally occurring protein that is modified to have at least one of an increased fidelity, thermostability, processivity and DNA-RNA substrate affinity, e.g. as described in Baranauskas et al (Protein Eng Des Set, 2012; Arezi B and Hogrefe H (Nucleic Acids Res, 2009; 37(2):473-81) and/or a reverse transcriptase that lacks ribonuclease H activity, e.g. as described in Kotewicz et al (Nucleic Acids Res, 1988; 16(1): 265-277). The reverse transcriptase for use in the invention may be mesophilic or thermophilic.

The reverse transcriptase for use in the method of the invention may be derived from a virus, preferably a retrovirus. The reverse transcriptase may be selected from the group consisting of Superscript II reverse transcriptase, Maxima reverse transcriptase, Protoscript II reverse transcriptase, moloney murine leukemia virus reverse transcriptase (MMLV-RT), HighScriber reverse transcriptase, avian myeloblastosis virus (AMV) reverse transcriptase, human immunodeficiency virus type 1 reverse transcriptase, human T-cell leukemia virus type 1 reverse transcriptase (HTLV-1-RT), bovine leukemia virus reverse transcriptase (BLV-RT) and Rous Sarcoma Virus reverse transcriptase (RSV-RT). Preferably, the reverse transcriptase is selected from the group consisting of M-MLV RT (derived from the Moloney murine leukemia virus), HIV-1 RT (derived from the human immunodeficiency virus type 1), AMV RT (derived from the avian myeloblastosis virus), variants thereof, and engineered versions thereof. The reverse transcriptase may be an MMLV-RT, having one or more point mutations. A preferred MMLV-RT point mutation may be selected from the group consisting of D200N, L603W, T330P, T306K and W313F, e.g. as described in Anzalone et al (supra).

Optionally, the Reverse Transcriptase is obtainable from a yeast, including Saccharomyces, Neurospora, Drosophila; primates; and rodents. See, for example, Weiss, et al, U.S. Pat. No. 4,663,290 (1987); Gerard, G. R., DNA:271-79 (1986); Kotewicz, M. L., et al. Gene 35:249-58 (1985); Tanese, N., et al, Proc. Natl. Acad. Sci. (USA):4944-48 (1985); Roth, M. J., at al, J. Biol. Chem. 260:9326-35 (1985); Michel, F., et al. Nature 316:641-43 (1985); Akins, R. A., et al. Cell 47:505-16 (1986), EMBO J. 4:1267-75 (1985); and Fawcett, D. F., Cell 47:1007-15 (1986) (each of which are incorporated herein by reference in their entireties).

Exemplary reverse transcriptases for use in the present invention include, but are not limited to, Moloney Murine Leukemia Virus (M-MLV); Human Immunodeficiency Virus (HIV) reverse transcriptase and avian Sarcoma-Leukosis Virus (ASLV) reverse transcriptase, which includes but is not limited to Rous Sarcoma Virus (RSV) reverse transcriptase. Avian Myeloblastosis Virus (AMV) reverse transcriptase, Avian Erythroblastosis Virus (AEV), Helper Virus MCAV reverse transcriptase, Avian Myelocytomatosis Virus MC29 Helper Virus MCAV reverse transcriptase. Avian Reticuloendotheliosis Virus (REV-T) Helper Virus REV-A reverse transcriptase, Avian Sarcoma Vims UR2 Helper Virus UR2AV reverse transcriptase. Avian Sarcoma Virus Y73 Helper Virus YAV reverse transcriptase, Rous Associated Virus (RAV) reverse transcriptase, Myeloblastosis Associated Virus (MAV) reverse transcriptase, Feline Leukemia Virus reverse transcriptase, Cauliflower mosaic virus Reverse transcriptase, Klebsiella pneumonia Reverse transcriptase, Escherichia coli Reverse transcriptase, Bacillus subtilis Reverse transcriptase, Eubacterium rectale Reverse transcriptase and Geobacillus stearothermophilus Reverse transcriptase.

The reverse transcriptase may be a variant of a wild type reverse transcriptase, preferably comprising a mutation that impacts or changes one or more enzymatic activities (e.g., RNA-dependent DNA polymerase activity, RNase H activity, or DNA/RNA hybrid-binding activity) and/or an enzyme property (e.g., thermostability, processivity, or fidelity). In addition or alternatively, the reverse transcriptase (RT) may comprise one or more mutations which render the RT more or less stable, less prone to aggregration, and/or facilitates purification and/or detection, and/or other the modification of properties or characteristics.

Preferably the reverse transcriptase has a high fidelity, preferably having an error-rate that is less than one error in 15,000 nucleotides synthesized.

It is understood herein that a CRISPR-nuclease, preferably a CRISPR-nuclease as defined herein, and a reverse transcriptase, preferably a reverse transcriptase as defined herein, for use in the method may be separate entities, i.e. are separate proteins. Alternatively, the CRISPR-nuclease and reverse transcriptase, preferably the CRISPR nuclease and/or reverse transcriptase as defined herein, used in the method of the invention are fused together, i.e. constitute a fusion protein. Preferably, the reverse transcriptase is fused to the C-terminus of the CRISPR-nuclease, preferably using a linker, preferably a flexible linker, between the CRISPR nuclease and Reverse Transcriptase.

### Template RNA Molecule

A template RNA molecule can be any RNA molecule that enables a reverse transcriptase to label the free 3′ end of a target nucleic acid fragment. To this end, the template RNA molecule may direct the reverse transcriptase to the free 3′-end of the target nucleic acid fragment and preferably functions as a template for the addition of additional nucleotides to the free 3′-end.

The size of the template RNA molecule can vary and may be dependent on the number of nucleotides added to the 3′ end of the target nucleic acid fragment. The size of the template RNA molecule is preferably between about 5-500 nt, 10-250 nt, 15-200 nt, 20-150 nt, 25-100 nt, or between about 30-50 nt. The size of the template RNA molecule can be 25, 26, 27, 28, 29, 30, 31, 32, 33, 34, 35, 36, 37, 38, 39, 40 or more nucleotides.

The template RNA for use in the method of the invention preferably comprises a binding domain and a template domain. The template RNA molecule may consist of a binding domain and a template domain. Preferably, the binding domain is located at the 3′-end of the template RNA molecule and the template domain is located at the 5′-end of the template RNA molecule.

The binding domain binds or “hybridizes” to the double-stranded nucleic acid molecule and can direct the reverse transcriptase to a free 3′-end of the target nucleic acid fragment. The size of the binding domain can be equal or substantially equal to the size of the template domain. The binding domain of the template RNA preferably comprises a sufficient number of nucleotides to hybridize the template RNA to the double-stranded nucleic acid molecule. The size of the binding domain is preferably between about 5-200 nt, 8-100 nt, 10-50 nt, 12-50 nt, 14-30 nt, or between about 15-20 nt. The size of the binding domain of the template RNA molecule is preferably 10, 11, 12, 13, 14, 15, 16, 17, 18, 19, 20, 21, 22, 23, 24, 25 or more nucleotides. The binding domain of the template RNA molecule preferably comprises a sequence that can anneal to a sequence at the 3′ end of the first or second strand of the target nucleic acid fragment. Hence, the nucleotide sequence of the binding domain is preferably complementary to a sequence in the target nucleic acid fragment. The nucleotide sequence is preferably complementary to a sequence located upstream, preferably located immediately upstream, of the free 3′-end of the target nucleic acid fragment. Preferably, the nucleotide sequence of the binding domain is at least 60%, 65%, 70%, 75%, 80%, 85%, 90%, 95%, 96%, 97%, 98%, 99% or 100% complementary to a sequence located upstream, preferably located immediately upstream, of the free 3′ end of the target nucleic acid. Put differently, the binding domain of a template RNA molecule used for labelling the free 3′-end of the first strand of the target nucleic acid fragment preferably comprises a sequence having at least 60%, 65%, 70%, 75%, 80%, 85%, 90%, 95%, 96%, 97%, 98%, 99% or 100% sequence identity with the sequence located immediately 3′ of the generated 5′ end of the second strand of the target nucleic acid fragment. Alternatively or in addition, the binding domain of a template RNA molecule used for labelling the free 3′-end of the second strand of the target nucleic acid fragment preferably comprises a sequence having at least 60%, 65%, 70%, 75%, 80%, 85%, 90%, 95%, 96%, 97%, 98%, 99% or 100% sequence identity with the sequence located immediately 3′ of the generated 5′ end of the first strand of the target nucleic acid fragment. The nucleotide sequence of the binding domain of the target RNA molecule may comprise a sequence that is partly or fully complementary the sequence in the crRNA. The binding domain may comprise a sequence of about 10, 11, 12, 13, 14, 15, 16, 17, 18, 19 or 20 nucleotides that is partly or fully complementary to the sequence of the crRNA used for guiding a CRISPR-nuclease complex as defined herein. The sequence that can be bound or “targeted” by the binding domain of the template RNA may be present once in the double-stranded nucleic acid molecule. Alternatively, the sequence may be present at least 2, 3, 4, 5, 10 times or more often.

In addition to the binding domain, the template RNA molecule preferably also comprises a template domain adjacent, preferably directly adjacent, to the binding domain. The template domain aids in the addition of one or more nucleotides at the free 3′ end of the target nucleic acid fragment by functioning as template for the reverse transcriptase. The sequence of the template domain thus determines the sequence and the number of the nucleotides added to the free 3′-end of the target nucleic acid fragment. The sequence of the newly added nucleotides may be the reverse complement of the sequence of the template domain. The size of the template domain is preferably between about 1-200 nt, 5-100 nt, 10-50 nt, 12-40 nt, 14-30 nt, or between about 15-20 nt. The size of the template domain of the template RNA molecule is preferably 1, 2, 3, 4, 5, 6, 7, 8, 9, 10, 11, 12, 13, 14, 15, 16, 17, 18, 19, 20, 21, 22, 23, 24, 25 or more nucleotides. The template domain of the RNA molecule may comprise or consist of a functional domain, preferably selected from the group consisting of a sequencing primer binding site, an amplification primer binding site, a barcode and a UMI, or a combination thereof. The template domain may comprise a sequencing primer binding site and a barcode. Alternatively or in addition, the template domain may comprise at least one of an amplification primer binding site and a sequencing primer binding site, in addition to at least one of a barcode and an UMI. Preferably the (amplification and/or sequencing) primer binding site is located 5′ of the barcode in the template domain of the template RNA. Preferably the (amplification and/or sequencing) primer binding site is located 5′ of the UMI in the template domain of the template RNA. Preferably, the template domain comprises in a 5′ to 3′ direction an amplification primer binding site, a sequencing primer binding site and a barcode and/or a UMI.

The template RNA may comprise the following order of elements in a 5′ to 3′ direction: a (amplification and/or sequencing) primer binding site, a barcode, and a binding domain, wherein the primer binding site and the barcode are comprised in the template domain.

Alternatively or in addition, the template RNA may comprise the following order of elements in a 5′ to 3′ direction: a (amplification and/or sequencing) primer binding site, a UMI, and a binding domain, wherein the primer binding site and the UMI are comprised in the template domain.

The template RNA molecule and the guide RNA may be separate entities. Preferably, the template RNA and the crRNA, and optionally the tracrRNA are separate RNA molecules. As a non-limiting example, a plurality of samples comprising a nucleic acid molecule is provided in step a) and in step b) a double-stranded break is generated at the same position in each nucleic acid molecule by using the same guide RNA. In step d) the plurality of samples may subsequently be contacted by a plurality of template RNA molecules, wherein e.g. each template RNA molecule generates a unique label at the free 3′-end of each nucleic acid molecule.

Alternatively, the template RNA molecule and guide RNA molecule are covalently bound, i.e. form a single RNA molecule. Preferably, the template RNA molecule is located at the 3′-end of the RNA molecule and the guide RNA is located at the 5′-end of the RNA molecule. The template RNA may be located directly adjacent to the guide RNA in a single molecule. Alternatively, the template RNA may be separated from the from the guide RNA by one or more, naturally or non-naturally-occurring, nucleotides.

Optionally the plurality of samples are processed in parallel in step a)-e), preferably in separate reaction vessels.

### Chemical Modifications of the RNA Molecules

The RNA molecules used in the method of the invention include at least one of a guide RNA and template RNA. The guide RNA may comprise at least one of a sgRNA, crRNA and a tracrRNA. The template RNA may be fused to the guide RNA.

At least one of the RNA molecules used in the method of the invention may comprise or consist of non-modified or naturally occurring nucleotides. Optionally, all RNA molecules used in the method of the invention may comprise or consist of non-modified or naturally occurring nucleotides.

Alternatively or in addition, the at least one of the RNA molecules used in the method of the invention may comprise or consist of modified or non-naturally occurring nucleotides. Optionally, all RNA molecules used in the method of the invention may comprise or consist of modified or non-naturally occurring nucleotides. Such chemically modified nucleotides preferably protect the RNA molecule, or molecules, against degradation. Optionally, the at least one of the RNA molecules, i.e. at least on the guide RNA and the template RNA, comprises ribonucleotides and non-ribonucleotides. At least one of the RNA molecules may comprise one or more ribonucleotides and one or more deoxyribonucleotides.

Optionally, the at least one of the RNA molecules, i.e. at least on the guide RNA and the template RNA, comprises one or more non-naturally occurring nucleotides or nucleotide analogues, such as a nucleotide with phosphorothioate linkage, a locked nucleic acid (LNA) nucleotides comprising a methylene bridge between the 2′ and 4′ carbons of the ribose ring, bridged nucleic acids (BNA), 2′-0-methyl analogues, 2′-deoxy analogues, 2′-fluoro analogues or combinations thereof. The modified nucleotides may comprise modified bases selected from the group consisting of, but not limited to, 2-aminopurine, 5-bromo-uridine, pseudouridine, inosine, and 7-methylguanosine.

At least one of the RNA molecules, i.e. at least one of the guide RNA and template RNA, may be chemically modified by incorporation of 2′-0-methyl (M), 2′-0-methyl 3′phosphorothioate (MS), 2′-0-methyl 3′thioPACE (phosphonoacetate) (MSP), or a combination thereof, at one or more terminal nucleotides. Such chemically modified RNAs can comprise increased stability and/or increased activity as compared to unmodified RNAs. (Hendel et al, 2015, Nat Biotechnol. 33(9);985-989). In an embodiment of the invention, deoxyribonucleotides and/or nucleotide analogues can be incorporated in the engineered RNA structures.

In an embodiment, the first and optionally second labelled strand may directly serve as a template(s) for further processing such as amplification and/or sequencing in case said label(s) comprise(s) functional domains required for such further processing such as an amplification and/or sequencing primer binding site. In another embodiment, said first and optionally second labelled strand are first extended and/or annealed to introduce such functional domains for further processing such as amplification and/or sequencing as further indicated herein below. In an optional embodiment, the labelled strands are amplified using one or more tailed primers comprising functional elements such as a UMI and/or a (sample) barcode. In addition or alternatively, said one or more tailed primers may comprise one or more sequencing primer binding sites for in order to sequence the resulting (barcoded) amplicons.

### Annealing an Oligonucleotide—Step f)

The method of the invention may comprise a step of further extending the generated label. Such extension may thus further increase the size of the label attached to the target nucleic acid fragment. Preferably, this further extension step makes use of the label generated in step c) and/or step e) as detailed herein.

This step is not limited to any particular method, and the skilled person may use any conventional method for further extending the target nucleic acid fragment. Preferably, this further extension step may comprise at least one of:
i) Amplifying the target nucleic acid fragment, wherein at least one of the amplification primers at least partly anneals to the generated label; and
ii) Annealing an oligonucleotide to the labelled 3′-end of the strand of the target nucleic acid fragment.

Prior to further extending the target nucleic acid fragment, the RNA molecules, such as the template RNA and/or the guide RNA, may be degraded. Thus prior to further extending the label of the target nucleic acid fragment, at least one of the template RNA and guide RNA may be degraded. The invention is not limited to any particular RNA degradation step and the skilled person can use any conventional means to degrade the RNA. The RNA is preferably degraded using a ribonuclease (RNAse), preferably an endonuclease, such as, but not limited to, RNAse H. Preferably, the RNA is degraded using an RNAse H. The RNA degradation is preferably performed under experimental conditions wherein the RNAse is capable of degrading at least one of the guide RNA and the template RNA, i.e. under experimental conditions wherein the RNAse shows enzymatic activity. Such experimental conditions are well-known by the skilled person and/or can be determined using any conventional means. These experimental conditions may be dependent on the type of RNAse, as will be known to the skilled person. The experimental conditions can be the same or similar as the conditions described in the experimental section below.

The primers for amplification of the nucleic acid fragment may hybridize solely to at least part of the label, or at least one of the primers may hybridize to both at least part of the label and to one or more nucleotides of the target nucleic acid fragment. Hence, at least one of the primers may be used for selective amplification.

Optionally, at least one of the amplification primers may comprise a functional domain, preferably selected from the group consisting of a restriction site domain, a capture domain, a sequencing primer binding site, an amplification primer binding site, a detection domain, a barcode sequence, a transcription promoter domain and a PAM sequence, or any combination thereof. The barcode can be, but is not limited to, a sample barcode.

The label may be extended in step f) by annealing a first oligonucleotide to the labelled 3′-end of the first strand of the target nucleic acid fragment. The oligonucleotide preferably specifically hybridizes to the labelled 3′-end of the first strand of the target nucleic acid fragment. Step f) may further comprise the annealing a second oligonucleotide to the labelled 3′-end of the second strand. The same oligonucleotide may anneal to both the label at the 3′-end of the first strand and the label at the 3′-end of the second strand, e.g. when the sequence of the label at the 3′-end of the first strand is identical, or nearly identical, to the nucleotide sequence of the label at the 3′-end of the second strand. Alternatively, the oligonucleotide annealing to the labelled 3′-end of the first strand is not capable of annealing to the, optionally labelled, 3′-end of the second strand under normal hybridizing conditions. Similarly, the oligonucleotide annealing to the labelled 3′-end of the second strand is not capable of annealing to the, optionally labelled, 3′-end of the first strand under normal hybridizing conditions. Thus preferably, the sequence of the label at the 3′-end of the first strand differs from the nucleotide sequence of the label at the 3′-end of the second strand to such extent that different oligonucleotides can be annealed at each side of the target nucleic acid fragment. Thus by varying the sequence of the generated labels, specific oligonucleotides can anneal to the target nucleic acid fragments.

The sequence of the oligonucleotide annealing to the labelled 3′-end of the first strand may be identical to the sequence of the oligonucleotide annealing to the labelled 3′-end of the second strand. In this embodiment, the sequence of the label extending the 3′-end of the first strand is thus preferably identical, or nearly identical, to the sequence of the label extending the 3′-end of the second strand.

Optionally, the sequence of the oligonucleotide annealing to the labelled 3′-end of the first strand may be identical to the sequence of the oligonucleotide annealing to the labelled 3′-end of the second strand, with the exception of the part of the oligonucleotide that can anneal to the generated label. In this embodiment, the sequence of the label extending the 3′-end of the first strand thus differs from the sequence of the label extending the 3′-end of the second strand.

Alternatively, the sequence extending the label at the 3′-end of the first strand and the sequence extending the label at the 3′-end of the second strand differ by one or more nucleotides.

It is understood herein that one could e.g. design a specific label for each DNA sample, and/or create specific labels for each target nucleic acid fragment, and/or specific labels for each site of a single target nucleic acid fragment, e.g. a specific label produced at the 3′ end of the first strand and another label produced at the 3′-end of the complementary strand of a single target nucleic acid fragment. Hence, the method as detailed herein provides for a versatile platform, wherein the produced labels can be straightforwardly customized to the particular needs of the experiment.

The oligonucleotide for use in the method of the invention has preferably at least one domain that can hybridize or “anneal” to the label produced in step c) and/or step e). This domain preferably has the same, or substantially the same, sequence as the template domain of the template RNA molecule. Optionally, the oligonucleotide consists of said domain hybridizing or annealing to the label. Alternatively, the oligonucleotide comprises a further functional domain or “tail”, preferably selected from the group consisting of a restriction site domain, a capture domain, a sequencing primer binding site, an amplification primer binding site, a detection domain, a barcode sequence, a transcription promoter domain and a PAM sequence, or any combination thereof. Preferably, the oligonucleotide comprises at least one of an UMI, a barcode and a primer binding site. The barcode can be, but is not limited to, a sample barcode, or a unique molecular identifier (UMI). Said further functional domain or “tail” is to be understood herein as a part of the oligonucleotide that does not hybridize or anneal to the label produced in step c) and/or step e). Optionally, the first and second oligonucleotide comprise a functional domain. The functional domain(s) located in the first and second oligonucleotide may be the same functional domain or different functional domains. The functional domains and the positions of these domains may be the same as described herein above for the functional domains optionally located in the first and second label. As an non-limiting example, the functional domains located in the first and second oligonucleotide may be used for amplification and/or sequencing and e.g. a barcode located in the first oligonucleotide and a barcode located in the second nucleotide together may form a combinatorial barcode.

Optionally, the domain hybridizing or annealing to the label has the same length as the length of the single-stranded label. Annealing the oligonucleotide to the label will thus result in a double-stranded label.

Optionally, the domain hybridizing or annealing to the label is one or a more nucleotides longer than the length of the single-stranded label. Annealing the oligonucleotide to the label results in a single-stranded overhang of one or more nucleotides, preferably an A- or T-overhang. Likewise, the oligonucleotide may be one or a more nucleotides shorter than the single-stranded label. Annealing the oligonucleotide to the label results in a single-stranded overhang of one or more nucleotides, preferably an A- or T-overhang, of the opposite strand.

Optionally, the domain hybridizing or annealing to the label is substantially shorter than the label and wherein a fill-in or PCR reaction is used to generate a double-stranded label.

Preferably, the oligonucleotide is a single-stranded adapter, preferably an adapter as defined herein above. The annealed oligonucleotide can be converted into a partly or fully double-stranded sequence. Said double-stranded sequence can be a double-stranded adapter. The adapter may be, or may be ligated to, a sequencing adapter, e.g. comprise a functional domain that allows for Roche 454A and 454B sequencing, ILLUMINA™ SOLEXA™ sequencing, Applied Biosystems' SOLID™ sequencing, the Pacific Biosciences' SMRT™ sequencing, Pollonator Polony sequencing, Oxford Nanopore Technologies or the Complete Genomics sequencing.

Optionally, the oligonucleotide annealing to the generated label, or labels, can have a partly or fully double-stranded structure, e.g. it forms a hairpin or stem loop structure.

Alternatively or in addition, a partly or fully double-stranded nucleic acid molecule may be annealed to the generated label, or labels. Such double-stranded nucleic acid may be a double-stranded adapter, or a cloning plasmid. The double-stranded adapter or cloning plasmid preferably comprises a single-stranded overhang that can hybridize to the generated label. The overhang is preferably a 3′-overhang. The other end of the double-stranded adapter, or cloning plasmid, preferably the 5′-end, preferably cannot hybridize to the generated label. In addition or alternatively, the other end of the double-stranded adapter, or cloning plasmid, preferably the 5′-end, cannot be ligated to the 3′-end of the double-stranded adaptor or cloning plasmid, and/or cannot be ligated to another adapter. Put differently, preferably the overhangs of the double-stranded adapter are designed to avoid adapter-adapter-ligations. Preferably, the double-stranded adapter, or cloning plasmid, comprises a 3′-end that can be ligated to a generated label and 5′-end that is blunt or comprises a single-nucleotide overhang, such as an A-overhang. The overhang at the 3′-end may be an 3′-overhang of the first strand. The overhang at the 5′-end, may be an 3′-overhang of the second strand.

The oligonucleotide may comprise one or more chemical moieties that protect against exonuclease digestion. Such moieties are preferably present in the 5′-terminal portion of the oligonucleotide. Such protective moieties may be phosphorothioates, which are known in the art to protect against nucleases. For instance phosphorothioates at the 5′-termini will prevent exonuclease degradation by a 5′ to 3′ exonuclease, such as T7 or lambda exonuclease. The 5′-terminal end of an oligonucleotide may comprise at least 1, 2, 3, 4, 5, 6, 7, 8, 9 or 10 phosphorothioate (PS) bonds. A PS bond substitutes a sulfur atom for a non-bridging oxygen in the phosphate backbone of an oligonucleotide, which renders the internucleotide linkage resistant to nuclease degradation. Alternatively or in addition, one or more chemical moieties may be incorporated in the label during step c) and/or step e), wherein said chemical moieties protect the nucleic acid against exonuclease digestion.

The method of the invention may thus further comprise a step of exonuclease treatment. Preferably the exonuclease treatment may be included in the method of the invention when the annealed oligonucleotide and/or the label comprises one or more chemical moieties that protect against exonuclease digestion. Alternatively or in addition, an exonuclease treatment step may be included after the reverse transcription in step c) and/or after step e). Alternatively or in addition, an exonuclease step may be included after cleavage of the double-stranded nucleic acid molecule in step b) and/or after step d). Preferably the exonuclease is inactivated after exonuclease treatment. As a non-limiting example, a thermostable Cas9 may be used in step b) and or step d), which preferably remains stable at temperatures between 60° C.-75° C. A subsequent exonuclease treatment step may be performed with an exonuclease that is unstable at elevated temperatures, e.g. that is unstable at a temperature between 60° C.-75° C. After exonuclease treatment at a suitable temperature, such as room temperature, the temperature may be elevated to inactive the exonuclease but not the (still bound) thermostable Cas9, such as elevating the temperature to between 60° C.-75° C. After inactivating the exonuclease, the subsequent reverse transcriptase step may be performed.

### Ligating and/or Filling in Reaction—Step g)

The method of the invention may further comprise a step g) of ligating the annealed oligonucleotide(s) to the target nucleic acid fragment and/or filing in the single-stranded overhang(s). Such single-stranded overhang(s) may be generated due to addition of a label at the free 3′-end of the target nucleic acid fragment and/or due to the annealing of the single-stranded oligonucleotide to the generated label.

The ligation step can be performed using any conventional means. The oligonucleotide may be ligated to the target nucleic acid fragment using any conventional ligase enzyme.

The ligation step g) is preferably performed under experimental conditions wherein the ligase enzyme is capable of ligating the annealed oligonucleotide(s) to target nucleic acid fragment, i.e. under experimental conditions wherein the ligase shows enzymatic activity. Such experimental conditions are well-known by the skilled person and/or can be determined using any conventional means. These experimental conditions may be dependent on the type of ligase, as will be known to the skilled person. The experimental conditions can be the same or similar as the conditions described in the experimental section below.

In case a single-stranded oligonucleotide is annealed to a label generated at the 3′-end of the first strand, the oligonucleotide is ligated to the 5′-end of the second strand. Similarly, in case a single-stranded oligonucleotide is annealed to a label generated at the 3′-end of the second strand, the oligonucleotide is ligated to the 5′-end of the first strand.

The filing in reaction, i.e. to generate a double-stranded DNA molecule, can be performed using any conventional means, such as using a DNA polymerase.

The filling-in reaction in step g) is preferably performed under experimental conditions wherein the polymerase is capable of filling in the single-stranded overhang generated by the annealed oligonucleotide(s), i.e. under experimental conditions wherein the polymerase shows enzymatic activity. Such experimental conditions are well-known by the skilled person and/or can be determined using any conventional means. These experimental conditions may be dependent on the type of polymerase, as will be known to the skilled person. The experimental conditions can be the same or similar as the conditions described in the experimental section below. These experimental conditions preferably at least include the presence of nucleotides, preferably naturally occurring nucleotides, preferably these experimental conditions include the presence of dNTPs, preferably at least one of adenine, guanine, cytosine and thymidine and optionally uracil.

Preferably, the ligation and filling-in step may be combined in a single reaction, e.g. by using a DNA repair mix, such as, but not limited to, the NEBNext®FFPE DNA Repair mix.

The single-stranded oligonucleotide or the at least partly double-stranded nucleic acid annealed and ligated to the label may comprise a primer binding site for subsequent amplification of the target nucleic acid fragment.

Alternatively or in addition, the oligonucleotide annealed and ligated to the label may be filled in to form a double-stranded sequence. Alternatively, a partly or fully double-stranded nucleic acid molecule may be annealed and ligated to the generated label. The, optionally double-stranded, sequence extending the label may be an adapter.

An “extended label” is understood herein as the sequence extending the target nucleic acid fragment that is obtainable after step f) and g) as defined herein. Depending on the context, the term “label” may thus include the label obtainable after step c) and/or step e), as well as the label obtainable after step f) and g). In a subsequent step h) a sequencing adapter may be ligated to the extended label. Any conventional sequencing adapter known in the art may be suitable for use in the invention. Preferably, the sequencing adapter comprises an end that can be ligated to the free 3′- and/or free 5′-end end of the extended label, or labels. The sequencing adapter thus preferably comprises an end that is compatible to the free 3′- and/or free 5′-end of the extended label, or labels. The sequencing adapter may comprise a blunt end or a single-stranded overhang of one or more nucleotides. As a non-limiting example, in case a free end of the extended label comprises a 3′-A overhang, the sequencing adapter preferably comprises a 3′-T overhang. The sequencing adapter may comprise one end that is compatible with the free end of the extended label, and one end that cannot be ligated to at least one of the extended label and a sequencing adapter.

In an embodiment, the, optionally extended, label comprises a protelomerase recognition sequence, preferably a TeIN protelomerase recognition sequence.

A protelomerase recognition sequence is any DNA sequence whose presence in a DNA template allows for its conversion into a closed linear DNA by the enzymatic activity of protelomerase. In other words, the protelomerase recognition sequence is required for the cleavage and re-ligation of double-stranded DNA by protelomerase to form a covalently closed linear DNA. Typically, a protelomerase recognition sequence comprises a perfect palindromic sequence, i.e. a double-stranded DNA sequence having two-fold rotational symmetry.

The length of the perfect inverted repeat differs depending on the specific organism. In Borrelia burgdorferi, the perfect inverted repeat is 14 base pairs in length. In various mesophilic bacteriophages, the perfect inverted repeat is 22 base pairs or greater in length. Also, in some cases, e.g. E. coli N15, the central perfect inverted palindrome is flanked by inverted repeat sequences, i.e. forming part of a larger imperfect inverted palindrome.

A protelomerase recognition sequence as used in the invention preferably comprises a double-stranded palindromic (perfect inverted repeat) sequence of at least 14 base pairs in length.

Preferred perfect inverted repeat sequences include the sequence NCATNNTANNCGNNTANNATGN (SEQ ID NO: 37) and variants thereof. This sequence is a 22 base consensus sequence. As e.g. disclosed in WO2010/086626, base pairs of the perfect inverted repeat are conserved at certain positions, while flexibility in sequence is possible at other positions. Thus preferably, this sequence is a minimum consensus sequence for a perfect inverted repeat sequence for use with a protelomerase in the method of the present invention. The protelomerase recognition sequence may have a sequence as described in WO2010/086626, which is incorporated herein by reference.

Preferably, the protelomerase recognition sequence has at least 80%, 85%, 90%, 95%, 96%, 97%, 98%, 99% or 100% sequence identity with SEQ ID NO: 38. The sequence of SEQ ID NO: 38 is:

5′-TATCAGCACACAATTGCCCATTATACGCGCGT
ATAATGGACTATTGTGTGCTGATA-3′.

Preferably, the protelomerase cleaves the, optionally extended, label between positions 28-29 in the recognition sequence and closes the cleaved ends.

In case there is a protelomerase recognition site introduced into the, optionally extended, label, the method may further comprise a step of contacting the labelled target nucleic acid fragment with a protelomerase, preferably a TeIN protelomerase, to cleave and covalently close the cleaved end, resulting in a target nucleic acid fragment comprising a closed end.

In an embodiment, the target nucleic acid fragment comprises a single label having a protelomerase recognition site, i.e. only at the 3′-end of the first strand or only at the 3′-end of the second strand. After generating a double-stranded label, the protelomerase cleaves and closes one end of the target nucleic acid fragment. The other end of the, optionally labelled, target nucleic acid fragment remains open. As a non-limiting example, a sequencing adapter can be annealed and/or ligated to this open end.

In another embodiment, the target nucleic acid fragment comprises a label at the 3′-end and a label at the 5′-end and both labels comprise a protelomerase recognition site. Following the generation of a double-stranded label, the protelomerase can cleave and close both ends of the target nucleic acid fragment. The closed nucleic acid fragment is protected against exonuclease degradation.

A preferred protelomerase for use in the invention is a bacteriophage protelomerase. A protelomerase can be selected from the group consisting of:phiHAP-1 from Halomonas aquamarina, PY54 from Yersinia enterolytica, phiKO2 from Klebsiella oxytoca, VP882 from Vibrio sp. and NI 5 from Escherichia coli, or variants of any thereof. The protelomerase may have an amino acid sequence as disclosed in WO2010/086626, which is incorporated herein by reference.

The use of bacteriophage NI 5 (TeIN) protelomerase or a variant thereof is particularly preferred. A preferred protelomerase has a sequence of at least 80%, 85%, 90%, 95%, 96%, 97%, 98%, 99% or 100% sequence identity with SEQ ID NO: 39. Variants include homologues or mutants thereof. Mutants include truncations, substitutions or deletions with respect to the native sequence. A variant preferably produces closed linear DNA from a template comprising a protelomerase recognition sequence as described herein above.

Optionally, the sample is exposed to an exonuclease after contacting the labelled target nucleic acid fragment with a protelomerase. The closed target nucleic acid fragment will be protected against exonuclease digestion and the non-closed not-target nucleic acid fragments will be degraded.

The method of the invention may further comprise a step) wherein, optionally a subset of, the target nucleic acid fragments are cleaved by a first programmable nuclease or a first restriction endonuclease, wherein preferably the programmable nuclease is an RNA-guided CRISPR nuclease, rendering an opened nucleic acid fragment to which optionally an adapter is ligated or annealed.

### Sequencing Method—Steps (i), (ii) and (iii)

In a further aspect, the method of the invention pertains a method for sequencing one or more target nucleic acid fragments. The sequencing method is preferably a deep-sequencing method. The sequencing method preferably comprises at least the steps of:
obtaining one or more labelled target nucleic acid fragments as defined herein; and
determining at least part of the sequence of the one or more target nucleic acid fragments.

In an embodiment, the method for sequencing one or more target nucleic acid fragments comprises the steps of:
(i) obtaining one or more labelled target nucleic acid fragments by the steps of
   a) providing a sample comprising a double-stranded nucleic acid molecule, wherein the double-stranded nucleic acid molecule comprises the sequence of interest;
   b) contacting the double-stranded nucleic acid molecule with a site-specific nuclease to generate a double-stranded break, wherein the double-stranded break results in a free 3′-end of the first strand of the target nucleic acid fragment;
   c) contacting the cleaved nucleic acid molecule with a reverse transcriptase and a template RNA molecule, thereby labelling the free 3′-end of the first strand of the target nucleic acid fragment with one or more nucleotides, wherein optionally the site-specific nuclease in step b) and the reverse transcriptase in step c) are separate entities; and
(iii) determining at least part of the sequence of the one or more target nucleic acid fragments. Hence, the labelled target nucleic acid fragments in step (i) may be obtained by performing at least steps a), b) and c) as detailed herein. The labelled target nucleic acid fragments of step (i) may be obtained by performing steps:
Steps a), b), and c);
Steps a), b), c), d) and e);
Steps a), b), d), c) and e);
Steps a), b), c); f), and optionally step g)
Steps a), b), c), d) and e); f), and optionally step g); and/or
Steps a), b), d), c) and e); f), and optionally step g)
   It is further understood herein that steps b) and d) may be performed substantially simultaneously and/or steps c) and e) may be performed substantially simultaneously.

The labelled target nucleic acid fragments obtained in step (i) may thus comprise one or more oligonucleotides annealed to the labelled target nucleic acid fragments. In addition, these annealed oligonucleotides may optionally have been ligated to the target nucleic acid fragments and/or made double-stranded. As detailed herein, the oligonucleotides may be a single-stranded or double-stranded adapter.

The sequencing method further comprises a step of determining at least part of the sequence of the one or more target nucleic acid fragments. The target nucleic acid fragment(s) obtained in step (i) may be used in single-molecule, real-time sequencing reaction, e.g., SMRT® Sequencing from Pacific Biosciences, Menlo Park, Calif. The use of other sequencing technologies is also contemplated, e.g., nanopore sequencing (e.g., from Oxford Nanopore or Ontera), Solexa® sequencing (IIlumina), tSMS™ sequencing (Helicos), Ion Torrent® sequencing (Life Technologies), pyrosequencing (e.g., from Roche/454), SOLID® sequencing (Life Technologies), microarray sequencing (e.g., from Affymetrix), Sanger sequencing, DNB seg™ (MGI Tech Co., Ltd), etc. The sequencing method may be capable of sequencing, e.g., >200 nt or more. The sequencing method may be capable of sequencing long template molecules, e.g., >1000-10,000 bases or more. The sequencing method may be capable of detecting base modifications during a sequencing reaction, e.g., by monitoring the kinetics of the sequencing reaction. The sequencing method may analyze the sequence of a single template molecule, e.g., in real time. In a preferred embodiment, the prepared nucleic acid molecule library is sequenced by nanopore selective sequencing. In nanopore selective sequencing, during real time sequencing the generated data (either direct current signals or base calls translated from these current signals) is compared to one or more reference sequence(s). In case a set number of nucleotides or amount of signals of the target sequence align with the reference sequence, sequencing will proceed, if not, current is reversed thereby removing the nucleic acid from the pore and making the pore available for sequencing of a new nucleic acid. The set number of nucleotides may be at least the first 50, 60, 70, 80, 90, 100, 150, 200, 250, 300, 350, 400, 450, or 500 nucleotides of the nucleic acid read. The one or more reference sequences may be a multitude of different sequences. Preferably, the reference sequences are at least 50, 60, 70, 80, 90, 92, 93, 94, 95, 96, 97 98, 99 or 100% identical to a sequence of a target nucleic acid fragment obtained in steps a) - c), and optionally steps d) and e), of the method of the invention. In an embodiment, the reference sequences are at least 50, 60, 70, 80, 90, 92, 93, 94, 95, 96, 97 98, 99 or 100% identical to a particular subset of the one or more sequences of target nucleic acid fragments obtained in steps a)-c), and optionally steps d) and e), of the method of the invention. One of the benefits of selectively sequencing a particular subset by nanopore selective sequencing is that in different sequencing runs, different subsets may be sequenced using the prepared nucleic acid molecule library.

The sequencing method of the invention may further comprise a step (ii) of amplifying, preferably selectively amplifying, the one or more labelled target nucleic acid fragments.

The amplification reaction in step (ii) is preferably performed under experimental conditions wherein the (DNA) polymerase is capable of amplifying the one or more labelled target nucleic acid fragments, i.e. under experimental conditions wherein the polymerase shows enzymatic activity. Such experimental conditions are well-known by the skilled person and/or can be determined using any conventional means. These experimental conditions may be dependent on the type of polymerase, as will be known to the skilled person. These experimental conditions preferably at least include the presence of nucleotides, preferably naturally occurring nucleotides, preferably these experimental conditions include the presence of dNTPs, preferably at least one of adenine, guanine, cytosine and thymidine and optionally uracil.

Amplification can be performed using one or more primers annealing to only the label and/or annealing to only at least part of the annealed oligonucleotide. In addition or alternatively at least one of the primers may comprise at its 3′-end one or more nucleotides that can anneal to nucleotides present in the target nucleic acid fragment, i.e. for selective amplification. Hence in the latter case, at least one of the primers may comprise a sequence that can anneal to the label and/or that can anneal to at least part of the annealed oligonucleotide, in addition to one or more nucleotides at its 3′-end that can anneal to a sequence present in the target nucleic acid fragment. In addition or alternatively, one of the primers of the primer pair may anneal only to a sequence presents in the target nucleic acid fragment, i.e. is a so-called “nested” primer.

Optionally at least one of the primers of the primer pair comprises a functional domain, preferably selected from the group consisting of a restriction site domain, a capture domain, a sequencing primer binding site, an amplification primer binding site, a detection domain, a barcode sequence, a transcription promoter domain and a PAM sequence, or any combination thereof. The barcode can be, but is not limited to, a sample barcode.

The method for sequencing one or more target nucleic acid fragments may therefore comprise the steps of:
(i) obtaining one or more labelled target nucleic acid fragments by the steps of
   a) providing a sample comprising a double-stranded nucleic acid molecule, wherein the double-stranded nucleic acid molecule comprises the sequence of interest;
   b) contacting the double-stranded nucleic acid molecule with a site-specific nuclease to generate a double-stranded break, wherein the double-stranded break results in a free 3′-end of the first strand of the target nucleic acid fragment;
   c) contacting the cleaved nucleic acid molecule with a reverse transcriptase and a template RNA molecule, thereby labelling the free 3′-end of the first strand of the target nucleic acid fragment with one or more nucleotides, wherein optionally the site-specific nuclease in step b) and the reverse transcriptase in step c) are separate entities;
   d) optionally contacting the double-stranded nucleic acid molecule with a second site-specific nuclease to generate a second double-stranded break, wherein the second double-stranded break results in a free 3′-end of the second strand of the target nucleic acid fragment, wherein preferably step d) is performed simultaneously with step b);
   e) optionally contacting the target nucleic acid fragment with a reverse transcriptase and a second template RNA molecule, thereby labelling the second strand of the target nucleic acid fragment at the free 3′-end with one or more nucleotides, wherein preferably step e) is performed simultaneously with step c);
   f) optionally annealing a first oligonucleotide to the labelled 3′-end of the first strand of the target nucleic acid fragment, wherein optionally the template RNA and crRNA are degraded prior to annealing the first oligonucleotide;
   g) optionally ligating and/or filling in the annealed oligonucleotide(s);
(ii) optionally amplifying, preferably selectively amplifying, the one or more labelled target nucleic acid fragments; and
(iii) determining at least part of the sequence of the, optionally amplified, one or more target nucleic acid fragments.

Optionally, the method of the invention is multiplexed, i.e. applied simultaneously for multiple nucleic acid samples, such as for at least about 5, 10, 20, 30, 40, 50, 60, 70, 80, 90, 100, 500, 1000 or more nucleic acid samples. The method may be performed in parallel for multiple samples, wherein “in parallel” is to be understood herein as substantially simultaneously but each sample being processed in a separate reaction tube or vessel.

In addition or alternatively, one or more steps of the method of the invention may be performed on pooled samples. The pooling step may for example be after any one of steps a), b), c), d), e), f) and g), and/or after any one of steps (i) and (ii). Preferably, the pooling step is after at least one step f) and step g), and/or after at least one of step (i) and step (ii). Preferably, the pooling step is after step g) and/or after at least one of step (i) and (ii).

In order to trace back the enriched, isolated and/or sequenced fragments to the originating sample, the fragments may be tagged with an identifier prior to pooling the samples. Such identifier can be any detectable entity, such as, but not limited to, a radioactive or fluorescent label, but preferably is a particular nucleotide sequence or combination of nucleotide sequences, preferably of defined length. The identifier is preferably present in at least one of the label, the oligonucleotide annealing to the label and the primer for amplifying the target nucleic acid fragment.

In addition or alternatively, the samples can be pooled using a clever pooling strategy, such as, but not limited to, a 2D and 3D pooling strategy, such that after pooling each sample is encompassed in at least two or three pools, respectively. A particular target nucleic acid fragment can be traced back to the originating sample by using the coordinates of the respective pools comprising the particular enriched, isolated and/or sequenced target fragment.

### Further Aspects

In an aspect, the invention pertains to a labelled target nucleic acid fragment. The labelled target nucleic acid fragment can be obtainable by the method of the invention. The labelled target nucleic acid fragment may be obtainable by performing at least one of:
Steps a), b), and c);
Steps a), b), c), d) and e);
Steps a), b), d), c) and e);
Steps a), b), c); f), and optionally step g)
Steps a), b), c), d) and e); f), and optionally step g); and/or
Steps a), b), d), c) and e); f), and optionally step g)
   It is further understood herein that steps b) and d) may be performed substantially simultaneously and/or steps c) and e) may be performed substantially simultaneously.
   In another aspect, the invention concerns a sequencing library, preferably a deep-sequencing library, obtainable by the method of the invention. The deep-sequencing library may be obtainable by performing at least one of:
Steps a), b), and c);
Steps a), b), c), d) and e);
Steps a), b), d), c) and e);
Steps a), b), c); f), and optionally step g)
Steps a), b), c), d) and e); f), and optionally step g); and/or
Steps a), b), d), c) and e); f), and optionally step g)

In addition, step (ii) may be performed to amplify the sequencing library obtainable by the method of the invention.

In addition, the sequencing library comprises a collection of pooled labelled target nucleic acid fragments, preferably using a pooling strategy as defined herein. The labelled target nucleic acid fragments preferably comprise a barcode, preferably a sample barcode.

In another aspect, the invention relates to a construct for use in the method of the invention. The construct preferably comprises a sequence encoding a site-specific nuclease as defined herein and comprising a sequence encoding at least one of a reverse transcriptase and a template RNA molecule as defined herein. Alternatively, the construct may comprise a sequence encoding a reverse transcriptase and a sequence encoding a template RNA molecule as defined herein.

The construct may further comprise a sequence encoding a guide RNA. Preferably, the construct may further comprise a sequence encoding at least one of a sgRNA, crRNA and optionally a tracrRNA. The construct may comprise at least 2, 3, 4, 5, 6, 7, 8, 9, 10 or more template RNA molecules. In addition or alternatively, the construct may comprise at least 2, 3, 4, 5, 6, 7, 8, 9, 10 or more guide RNAs. The template RNA molecules and/or the guide RNA molecules may cleaved after transcription, e.g. by incorporating a cleavage site in between the template RNA molecules, in between the guide RNA and/or in between the template RNA and the guide RNA. A preferred cleavage site is a tRNA cleavage site, such as described in WO 2016/061481, which is incorporated herein by reference.

In an aspect, the invention pertains to a kit for carrying out the method of the invention. Preferably the kit comprises at least three components, wherein
the first component is a site-specific nuclease as defined herein, or construct encoding the same, and optionally at least one of a crRNA, tracrRNA and a sgRNA, or construct encoding the same, preferably a construct as defined herein;
the second component is a DNA polymerase, preferably a reverse transcriptase, as defined herein, or construct encoding the same; and
the third component is a template RNA molecule as defined herein, or construct encoding the same.
   In a preferred embodiment, the kit comprises at least two different crRNAs and/or sgRNAs for excision of at least one target fragment from a double-stranded nucleic acid molecule of a sample. In a further preferred embodiment, the kit comprise a set of pairs of crRNAs and/or sgRNAs for excision of a set of target fragments from a double-stranded nucleic acid molecule of a sample, wherein a set of pairs may be 2, 3, 4, 5, 6, 7, 8, 9, 10 or more, such as at least about 50, 100, 150, 200, 250, 300, 350, 400, 450, 500, 750, 1000 or more.

Optionally, said kit comprises at least one template RNA molecule for labelling one side of said at least one target fragment. The kit may further comprise a set of template RNA molecules for labelling a set of target fragments, wherein a set of template RNA molecules may be 2, 3, 4, 5, 6, 7, 8, 9, 10 or more, such as at least about 50, 100, 150, 200, 250, 300, 350, 400, 450, 500, 750, 1000 or more.

Alternatively or in addition, said kit comprises at least two template RNA molecules for labelling both sides of said at least one target fragment. The kit may further comprise a set of pairs of template RNA molecules for labelling both sides of a set of target fragments, wherein a set of pairs of template RNA molecules may be 2, 3, 4, 5, 6, 7, 8, 9, 10 or more, such as at least about 100, 150, 200, 250, 300, 350, 400, 450, 500, 750, 1000 or more.

In addition, the kit may further comprise at least one of a fourth, fifth, sixth and seventh component, wherein
the fourth component is one or more oligonucleotides as defined herein. Preferably the one or more oligonucleotides comprise at least one of a UMI, barcode and primer binding site;
the fifth component is one or more primers for selective amplification of a labelled target nucleic acid fragment, preferably one or more primers as defined herein;
the sixth component is one or more primers for non-selective (universal) amplification of the labelled target nucleic acid fragment, preferably one or more primers as defined herein; and
the seventh component is one or more primers for selective amplification of a subset of target nucleic acid fragments, preferably one or more primers as defined herein.
   The kit preferably comprises at least two or more guide RNAs and/or at least two or more template RNAs for processing multiple samples and/or multiple target nucleic acid fragments. The kit preferably comprises at least 2, 3, 4, 5, 6, 7, 8, 9, 10 or more guide RNAs and/or at least 2, 3, 4, 5, 6, 7, 8, 9, 10 or more template RNAs for processing multiple samples and/or multiple target nucleic acid fragments.

The components may be present in separate vials or combined in one or more vials.

Preferably, the volume of any of the vials within the kit do not exceed 100 mL, 50 mL, 20 mL, 5 mL, 4 mL, 3 mL, 2 mL or 1 mL.

The reagents may be present in lyophilized form, or in an appropriate buffer. The kit may also contain any other component necessary for carrying out the present invention, such as buffers, pipettes, microtiter plates and written instructions. Such other components for the kits of the invention are known to the skilled person.

### FIGURE LEGENDS

FIG. 1: Schematic representation of an embodiment of the invention. Step 1) Targeted position, step 2) Cas9 binding, step 3) Cas9 DNA cleavage, step 4) Cas9 binding and adapter RNA (herein further indicated as template RNA) annealing, step 5) Reverse transcription of the annealed RNA, and step 6) RNA degradation (and thus Cas9 and RT release). Annealing the DNA adapter may comprise a step 7A) DNA adapter annealing and step 7B) DNA adapter fill-in and ligation. Alternatively, annealing the DNA adapter may comprise a step 7) DNA adapter annealing and ligation

FIG. 2. A) Sequence of positions 5043-6074 of the lambda genome (SEQ ID NO: 8) B) Top: Fragment obtained after restriction with Cas9, labelling and annealing of the oligonucleotides (SEQ ID NO's: (9-24):. Bottom: Length of the Fragments obtained after amplification, the size of the different fragments and the primer sequences are indicated

FIG. 3. Expected (A) and obtained (B) results after amplification of the indicated fragments.

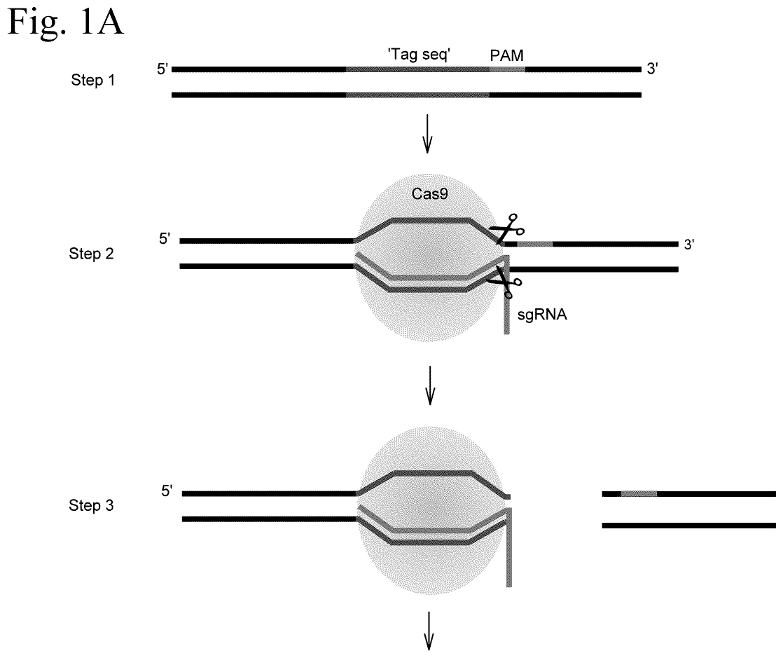

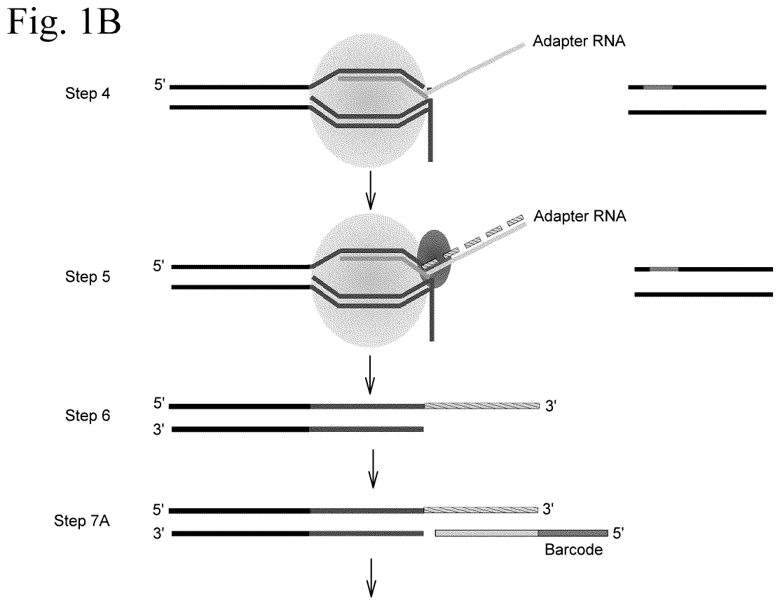

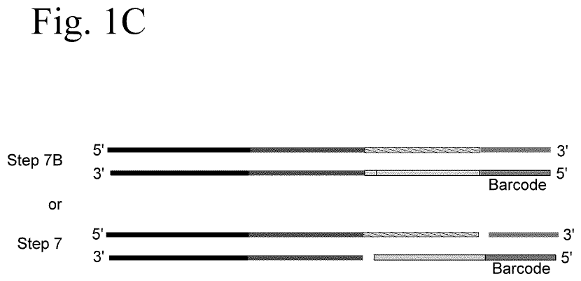

### EXAMPLES

### Example 1

### Materials and Methods

### Cas9 Cleavage of a Target Nucleic Acid Molecule

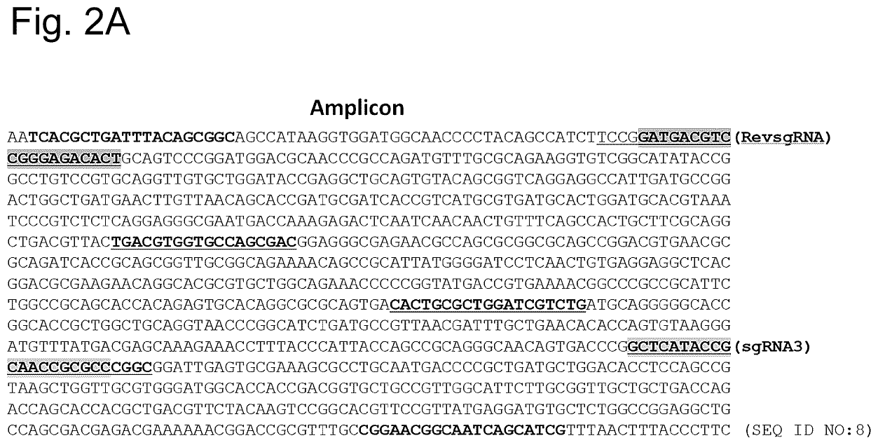

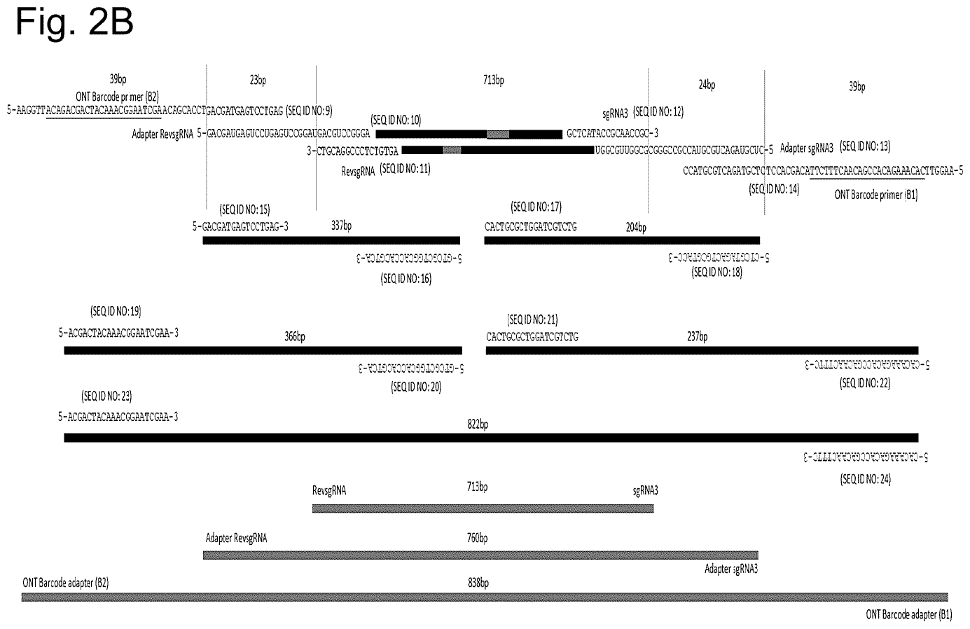

The double-stranded nucleic acid molecule was obtained by amplifying the positions 5043-6074 of the lambda genome using primers having SEQ ID NO: 25 or SEQ ID NO:26. The amplified λ DNA fragment (˜1030 bp) was subsequently cleaved with Cas9 at two selected locations, as indicated in FIG. 2, using the following reaction conditions:
Nuclease free water: 2.7 μl
10× Buffer 3.1 (NEB): 2 μl
3 uM RevsgRNA: 1.3 μl
3 uM sgRNA3: 1.3 μl
1 uM Cas9 Nuclease (NEB): 7.7 μl
Substrate DNA (100 ng/ul): 5 μl

### Total Volume: 20 μl

### Incubated at 37° C. for 1 h

Sequence RevsgRNA in a 5′ to 3′ direction (target sequence underlined):

(SEQ ID NO: 27)
AGUGUCUCCCGGACGUCAUCGUUUUAGAGC
UAGAAAUAGCAAGUUAAAAUAAGGCUAGUC
CGUUAUCAACUUGAAAAAGUGGCACCGAGU
CGGUGCUUUUUUU

Sequence sgRNA3 in a 5′ to 3′ direction (target sequence underlined):

(SEQ ID NO: 28)
GCUCAUACCGCAACCGCGCCGUUUUAGAGC
UAGAAAUAGCAAGUUAAAAUAAGGCUAGUC
CGUUAUCAACUUGAAAAAGUGGCACCGAGU
CGGUGCUUUUUUU

After cleavage, the DNA was either purified 3x and analyzed on a Bioanalyzer system (Agilent) or further processed as indicated below:
Reverse transcription of the cleaved DNA
   The cleaved DNA was subsequently extended at its 3′ end with a selected nucleotide sequence. To this end, the DNA was exposed to a reverse transcriptase and a first and second template RNA using the following reaction conditions:
Cleaved DNA: 15 μl
111.2 μM RevsgRNA-RNA-Ad: 0.7 μl
126.6 uM sgRNA3-RNA-Ad: 0.6 μl
5× Protoscript II buffer (NEB): 6 μl
Protoscript II RT (200 U/ul) (NEB): 1 μl
1M DTT: 0.3 μl
10 mM dNTPs: 1 μpl
MQ: 5.4 μl

### Total Volume: 30 μl

inccubated at 42° C. for 1 h, followed by an incubation at 65° C. for 20 min

Sequence of the first template RNA (RevsgRNA-RNA-Ad) in a 5′ to 3′ direction, (sequence hybridizing to the target DNA sequence is underlined, PAM sequence is Italic, and template sequence is in bold):

(SEQ ID NO: 29)
GACGAUGAGUCCUGAGUCCGGAUGACGUCCGGGA

Sequence of the second template RNA (sgRNA3-RNA-Ad) in a 5′ to 3′ direction, (sequence hybridizing to the target DNA sequence is underlined, PAM sequence is Italic, and template sequence is in bold):

(SEQ ID NO: 30)
CUCGUAGACUGCGUACCGCCGGGCGCGGUUGCGGU

### RNA Degradation

After the addition of the new sequence to the cleaved DNA, the RNA was degraded using an RNAse H treatment:
Extended DNA: 10 μl
10× RNAse H reaction Buffer (NEB): 10 μl
RNase H (5 U/ul) (NEB): 1 μl
MQ: 79 μl
Total Volume: 100 μl
Incubated at 37° C. for 20 min, followed by the addition of 1 μl 0.5M EDTA

### Adapter Annealing, Fill-In and Ligation

Finally, an oligonucleotide was annealed to the generated single-stranded overhang of the DNA molecule. As the overhang created using the first template RNA was different from the overhang created using the second template RNA, two different oligonucleotides were used. The annealed oligonucleotide was subsequently ligated to the DNA molecule and filled in (i.e. generating a double-stranded DNA molecule).
Sequence oligonucleotide in a 5′ to 3′ direction (RevsgRNA-BC2) (barcode underlined and sequence annealing to the overhang indicated in bold). This oligonucleotide can anneal to the overhang generated using RevsgRNA-RNA-Ad as a template RNA molecule:

(SEQ ID NO: 31)
AAGGTTACAGACGACTACAAACGGAATCGAA
CAGCACCTGACGATGAGTCCTGAG

Sequence oligonucleotide in a 5′ to 3′ direction (sgRNA3-BC1) (barcode underlined and sequence annealing to the overhang indicated in bold). This oligonucleotide can anneal to the overhang generated using sgRNA3-RNA-Ad as a template RNA molecule:

(SEQ ID NO: 32)
AAGGTTCACAAAGACACCGACAACTTTCTT
ACAGCACCTCTCGTAGACTGCGTACC

The oligonucleotides were annealed to the generated overhangs using the following reaction conditions:
Extended DNA: 10 μl
100 uM sgRNA3-BC1: 2.5 μl
100 uM RevsgRNA-BC2: 2.5 μl
FFPE DNA Repair Buffer (NEB): 3.25 μl
NEBNext FFPE DNA repair Mix (NEB): 1 μl
MQ: 11.75 μl
Total Volume 31 μl
Incubated at 20° C. for 15 min

### Amplification

To visualize the generated DNA products, different primer sets were used as indicated in FIG. 2 in a PCR reaction using standard conditions. The products generated after the Reverse Transcriptase reaction were amplified with the primer pair wherein the first primer was anneals to a sequence in the 2 DNA fragment, and the second primer anneals only to the newly generated overhang. Hence, the sequence of the second primer was GACGATGAGTCCTGAG (SEQ ID NO: 33) or CTCGTAGACTGCGTACC (SEQ ID NO: 34), generating an amplicon of respectively 337 bp or 204 bp.
In addition after annealing the oligonucleotides, the products were visualized using a standard PCR reaction with a primer pair, wherein the first primer could anneal to only a sequence present in the first oligonucleotide (RevsgRNA-BC2) and a second primer that could anneal to only a sequence present in the second oligonucleotide (sgRNA3-BC1). The sequences of these primers are ACGACTACAAACGGAATCGAA (SEQ ID NO: 35) and CACAAAGACACCGACAACTTTC (SEQ ID NO: 36) and the generated amplicon has an expected size of 822 bp.

### RESULTS AND CONCLUSIONS

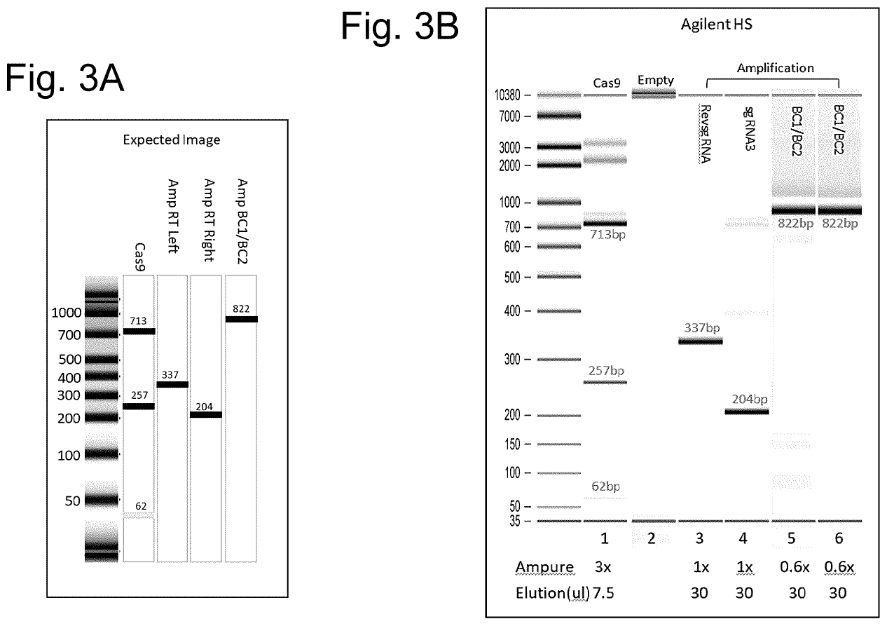

As shown in FIG. 3, clear amplification products were visible after amplification of the DNA fragments treated with the Cas9 complex and the Reverse transcriptase. The amplification products showed the expected size of 337 bp or 204 bp, confirming that the method as detailed herein can indeed extend the 3′ end of a DNA fragment of interest with a selective predetermined sequence.
The generated single-stranded overhangs can be used in downstream processes, e.g. to anneal oligonucleotides to the DNA fragment for subsequent deep-sequencing. Indeed, oligonucleotides could be straightforwardly annealed to the produced 3′ overhangs and the generated products were amplified, generating application products having the expected size of 822 bp (see FIG. 3).
By varying the sequence of the newly added single-stranded DNA, specific oligonucleotides can anneal to the generated overhangs. Indeed, this experiment shows that two different single-stranded overhangs could be created at each site of a cleaved DNA fragment, followed by annealing one oligonucleotide at one site of the fragment and another oligonucleotide at the other site of the DNA fragment.
One could e.g. design a specific overhang for each DNA sample, and/or create specific overhangs for each gene of interest, and/or specific overhangs for each site of a single gene (thus a specific overhang produced at the 3′ end of the first strand and another single-stranded overhang produced at the 3′-end of the complementary strand of a single gene). Hence, the method provides for a versatile platform, wherein the produced 3′-overhangs can be straightforwardly customized to the particular needs of the experiment.

1. A method for labelling a target nucleic acid fragment, wherein the target nucleic acid fragment comprises a first strand and a complementary second strand and wherein the target nucleic acid fragment comprises a sequence of interest, wherein the method comprises the steps of:
a) providing a sample comprising a double-stranded nucleic acid molecule, wherein the double-stranded nucleic acid molecule comprises the sequence of interest;
b) contacting the double-stranded nucleic acid molecule with a site-specific nuclease to generate a double-stranded break, wherein the double-stranded break results in a free 3′-end of the first strand of the target nucleic acid fragment; and
c) contacting the cleaved nucleic acid molecule with a DNA polymerase and a template molecule, preferably contacting the cleaved nucleic acid molecule with a reverse transcriptase and a template RNA molecule, thereby labelling the free 3′-end of the first strand of the target nucleic acid fragment with one or more nucleotides,
wherein optionally the site-specific nuclease in step b) and the reverse transcriptase in step c) are separate entities.

2. The method according to claim 1, wherein the method further comprises a step of:
d) contacting the double-stranded nucleic acid molecule with a second site-specific nuclease to generate a second double-stranded break, wherein the second double-stranded break results in a free 3′-end of the second strand of the target nucleic acid fragment, wherein preferably step d) is performed simultaneously with step b).

3. The method according to claim 2, wherein the method further comprises a step of:
e) contacting the target nucleic acid fragment with a DNA polymerase and a second template molecule, preferably with a reverse transcriptase and a second template RNA molecule, thereby labelling the second strand of the target nucleic acid fragment at the free 3′ -end with one or more nucleotides, wherein preferably step e) is performed simultaneously with step c).

4. The method according to claim 2, wherein the site-specific nuclease in step b) and/or step d) is a CRISPR-nuclease complex, preferably comprising at least one of a Cas9 or Cpf1 nuclease and a guide RNA.

5. The method according to claim 3, wherein the template RNA molecule of step c) comprises a sequence at its 3′ end that can anneal to a sequence at the 3′ end of the first strand of the target nucleic acid fragment, and wherein optionally the template RNA molecule of step e) comprises a sequence at its 3′ end that can anneal to a sequence at the 3′ end of the second strand of the target nucleic acid fragment.

6. The method according to claim 4, wherein the template RNA and the guide RNA are separate RNA molecules.

7. The method according to claim 1, wherein the sequence of the nucleotides extending the first strand differs from the sequence of the nucleotides extending the second strand of the target nucleic acid fragment, wherein preferably the one or more nucleotides extending the first and second strand have less than 90%, 80%, 60% or less than 40% nucleotide sequence identity.

8. The method according to claim 3, wherein the method further comprises a step of:
f) annealing a first oligonucleotide to the labelled 3′-end of the first strand of the target nucleic acid fragment, wherein optionally the template RNA and guide RNA are degraded prior to annealing the first oligonucleotide,
wherein preferably the oligonucleotide annealing to the labelled 3′-end of the first strand is not capable of annealing to the, optionally labelled, 3′-end of the second strand under normal hybridizing conditions.

9. The method according to claim 8, wherein step f) further comprises annealing a second oligonucleotide to the labelled 3′-end of the second strand, wherein preferably the oligonucleotide annealing to the labelled 3 ‘-end of the second strand is not capable of annealing to the, optionally labelled, 3’-end of the first strand under normal hybridizing conditions.

10. The method according to claim 8, wherein the method further comprises a step of:
g) ligating and/or filling in the annealed oligonucleotide(s).

11. The method according to claim 8, wherein at least one of the first and second oligonucleotide comprises at least one of an UMI, a barcode and a primer binding site.

12. A method for sequencing, preferably deep-sequencing, one or more target nucleic acid fragments, comprising the steps of:
(i) obtaining one or more labelled target nucleic acid fragments as defined in claim 1;
(ii) optionally amplifying, preferably selectively amplifying, the one or more labelled target nucleic acid fragments; and
(iii) determining at least part of the sequence of the, optionally amplified, one or more target nucleic acid fragments,
wherein preferably the one or more target nucleic acid fragments are obtained from one or more nucleic acid samples, and wherein optionally the one or more target nucleic acid fragments are pooled after step (i) and/or after step (ii).

13. A labelled target nucleic acid fragment obtainable by the method according to claim 1.

14. A construct encoding a site-specific nuclease and at least one of a reverse transcriptase and a template RNA molecule for use in a method according to claim 1, wherein the construct preferably further encodes a guide RNA.

15. A kit of parts comprising at least a first, second and third component for use in a method according to claim 1, wherein:
the first component is a site-specific nuclease, or construct encoding the same, and optionally a guide RNA, or construct encoding the same;
the second component is a reverse transcriptase, or construct encoding the same; and
the third component is a template RNA molecule, or construct encoding the same,
wherein the kit preferably further comprises at least one of a fourth, fifth, sixth and seventh component, wherein
the fourth component is one or more oligonucleotides capable of annealing to either the labelled 3′ end of the first or second strand but not capable of annealing to the labelled 3′ end of the other strand under normal hybridizing conditions, wherein the one or more oligonucleotides optionally comprise at least one of a UMI, barcode and primer binding site;
the fifth component is one or more primers for amplification of the labelled target nucleic acid fragment;
the sixth component is one or more primers for non-selective amplification of the labelled target nucleic acid fragment; and
the seventh component is one or more primers for selective amplification of a subset of target nucleic acid fragments.

16. A deep-sequencing library obtainable by the method according to claim 12.

17. A construct encoding a site-specific nuclease and at least one of a reverse transcriptase and a template RNA molecule for use in a method according to claim 12, wherein the construct preferably further encodes a guide RNA.

18. A kit of parts comprising at least a first, second and third component for use in a method according to claim 12, wherein:
the first component is a site-specific nuclease, or construct encoding the same, and optionally a guide RNA, or construct encoding the same;
the second component is a reverse transcriptase, or construct encoding the same; and
the third component is a template RNA molecule, or construct encoding the same,
wherein the kit preferably further comprises at least one of a fourth, fifth, sixth and seventh component, wherein
the fourth component is one or more oligonucleotides capable of annealing to either the labelled 3′ end of the first or second strand but not capable of annealing to the labelled 3′ end of the other strand under normal hybridizing conditions, wherein the one or more oligonucleotides optionally comprise at least one of a UMI, barcode and primer binding site;
the fifth component is one or more primers for amplification of a labelled target nucleic acid fragment as defined in claim 12;
the sixth component is one or more primers for non-selective amplification of the labelled target nucleic acid fragment; and
the seventh component is one or more primers for selective amplification of a subset of target nucleic acid fragments.
